(19) United States
(12) Patent Application Publication
(10) Pub. No.: US 2015/0142327 A1
(43) Pub. Date: May 21, 2015

(54) METHOD FOR IMPROVING THE ACCURACY OF CHEMICAL IDENTIFICATION IN A RECOGNITION-TUNNELING JUNCTION

(71) Applicant: ARIZONA BOARD OF REGENTS on behalf of ARIZONA STATE UNIVERSITY, Scottsdale, AZ (US)

(72) Inventors: Brian Alan ASHCROFT, Mesa, AZ (US); Stuart LINDSAY, Phoenix, AZ (US); John SHUMWAY, Tempe, AZ (US)

(21) Appl. No.: 14/493,961

(22) Filed: Sep. 23, 2014

Related U.S. Application Data
(63) Continuation-in-part of application No. PCT/US2013/032346, filed on Mar. 15, 2013.
(60) Provisional application No. 61/616,517, filed on Mar. 28, 2012.
(60) Provisional application No. 61/989,870, filed on May 7, 2014.

(51) Int. Cl. G01N 27/414; G01N 33/68

(52) U.S. Cl. CPC G01N 27/414; G01N 33/68
USPC 702/19

(57) ABSTRACT

A method to identify a chemical target trapped in a tunnel junction with a high probability of a correct assignment based on, a single read of the tunnel current signal. The method recognizes and rejects background signals produced in the absence of target molecules, and do so accurately without rejecting useful signals from the target molecules. The identity of signals generated by electron tunneling through an analyte is provided and comprises determining a plurality of characteristics of each signal current spike, generating one or more training signals with a set of analytes, where the analytes may comprise a first analyte, and using the training signals to find one or more boundaries in a space of dimension equal to one or more parameters, wherein the space is partitioned such that a signal from the first analyte of interest is separated from a signal from the second analyte of interest.

### CROSS REFERENCE TO RELATED APPLICATIONS

This application is a continuation-in-part (CIP) of PCT Application No. PCT/US2013/032346 filed Mar. 15, 2013, titled “METHOD FOR IMPROVING THE ACCURACY OF CHEMICAL IDENTIFICATION IN A RECOGNITION-TUNNELING JUNCTION”, which claims priority to U.S. Provisional Patent Application No. 61/616,517 filed Mar. 28, 2012, and entitled, “METHOD FOR IMPROVING THE ACCURACY OF CHEMICAL IDENTIFICATION IN A RECOGNITION-TUNNELING JUNCTION”. This application also claims priority to U.S. Provisional Patent Application No. 61/989,870, filed May 7, 2014 and entitled “SYSTEMS AND METHODS FOR CALLING SINGLE MOLECULE EVENTS WITH HIGH ACCURACY AND LIMITED PARAMETERS”, the entire disclosures of which are herein incorporated by reference in their entirety.

### STATEMENT REGARDING FEDERALLY SPONSORED RESEARCH & DEVELOPMENT

Inventions of this disclosure were made with government support under NIH Grant No. RO1 HG00623, awarded by the National Institute of Health. The U.S. Government has certain rights in inventions disclosed herein.

The application contains at least one drawing executed in color.

### FIELD OF THE DISCLOSURE

Embodiments of the present disclosure are directed to electronic identification of chemical species in a tunnel-junction device, and more particularly to a tunnel junction used as a readout for molecular sequencing.

### BACKGROUND

Reducing the cost of DNA sequencing below that of present “next generation” techniques will probably require the replacement of chemical methods, with associated reagent costs, by strictly physical means in which preparation of the DNA sample is the only chemical step (Zwolak and Di Ventra, 2008; Branton et al., 2008). Electron tunneling across a DNA molecule has been proposed (Zwolak and Di Ventra, 2005) and demonstrated (Tsutsui et al., 2010; Tsutsui et al., 2011) as a candidate base reading system. It is a possible alternative to ion-current sensing where individual nucleotides are readily recognized by the size the current blockage they produce (Clarke et al., 2009), but reading bases embedded within a polymer is still challenging (Derrington et al., 2010). Another approach, yet to be demonstrated in practice, is electronic modulation of the conductance of a graphene nanoribbon containing a nanopore. This might generate microamp signals, leading to very rapid sequencing (Saha et al., 2012).

### SUMMARY

Accordingly, some embodiments of the present disclosure provide a method to identify a chemical target trapped in a tunnel junction with a high probability of a correct assignment based on a single read of the tunnel current signal. It is a further object of some embodiments of the disclosure to additionally recognize and/or reject background signals produced in the absence of target molecules accurately, while limiting, and preferably eliminating rejections of useful signals from target molecules.

In some embodiments, a method of assigning the identity of signals generated by electron tunneling through an analyte is provided and comprises determining a plurality of characteristics of each signaVcurrent spike, generating one or more training signals with a set of analytes, where the analytes may comprise at least a first analyte and a second analyte, and using the training signals to find one or more boundaries in a space of dimension equal to one or more parameters, wherein the space is partitioned such that a signal from the first analyte of interest is separated from a signal from the second analyte of interest. The number of boundaries identified may be up to or equal to the number of parameters used in the method. In some embodiments, the set of analytes may contain any number of analytes. In some embodiments, the set of analytes contains 2, 3, 4, 5, 10, 15, or more analytes.

In some embodiments, the one or more parameters describe relationships between successive spikes. In some embodiments, the one or more parameters are obtained from a Fourier analysis of the spikes. In some embodiments, the one or more parameters are obtained from a Wavelet analysis of the spikes. In some embodiments, the one or more parameters are obtained from a Fourier analysis of clusters of spikes.

The analytes may be any analyte that is to be identified. In some embodiments, the analytes are DNA bases. In some embodiments, the analytes are modified DNA bases. In some embodiments, the analytes are amino acids. In some embodiments, the analytes are modified amino acids.

In some embodiments, the method may further comprise additional steps. In some embodiments, the method may further comprise weighting the calls by the frequency with which a call is repeated within a cluster of signals.

In some embodiments, a device is provided for determining the identity of one or more analytes in which a current versus time signal is characterized with three or more parameters.

In some embodiments, a computer system for assigning the identity of signals generated by electron tunneling through an analyte, comprising at least one processor, where the processor includes computer instructions operating thereon for performing the steps of a method for assigning the identity of signals generated by electron tunneling through an analyte according to any such method taught by the present disclosure.

In some embodiments, a computer system for determining the identity of one or more analytes is provided, and may comprise at least one processor, where the processor includes computer instructions operating thereon for performing the steps of a method for determining the identity of one or more analytes utilizing a current versus time signal having three or more parameters.

In some embodiments, a computer program for assigning the identity of signals generated by electron tunneling through an analyte is provided, and may comprise computer instructions for performing the steps of a method for assigning the identity of signals generated by electron tunneling through an analyte according to any such method taught by the present disclosure, and/or identifying one or more analytes utilizing a current versus time signal having three or more parameters.

In some embodiments, a computer readable medium containing a program is provided, where the program includes computer instructions for performing the steps of a method for assigning the identity of signals generated by electron tunneling through an analyte according to any such method taught by the present disclosure, and/or identifying one or more analytes utilizing a current versus time signal having three or more parameters.

In some embodiments, a method of assigning a chemical identity to a molecule signal is provided, and may comprise collecting signal data for at least two different molecules from a molecular identification or sequencing apparatus, the data including information corresponding to at least two signal parameters. The method may further comprise determining the distribution of the frequency of occurrence of the values of each of the parameters, and creating a plurality of at least three-dimensional plots, wherein each plot comprises the determined values for a pair of parameters, such that, the determined values for each parameter is plotted versus each of the other remaining parameters. The method may further comprise determining the separation of values between different analyte molecules for each of the plots, and selecting at least one plot of the plurality of plots which includes a separation of values between the two analyte molecules greater than a predetermined amount. The method may further comprise determining the identity of signals according to their determined value location on the selected plot.

In some embodiments, a method of assigning a chemical identity to a molecule signal is provided, and may comprise measuring a plurality of distributions of two or more signal parameters from signal data collected from a molecular identification or sequencing apparatus for known molecules. The method may further comprise determining at least one pair of parameters that best determine the separation of signals for identifying the known molecules. The method may further comprise using the pair of determined parameters, identifying one or more unknown molecules from second signal data collected from a molecular identification or sequencing apparatus.

In some embodiments, a system of assigning a chemical identity to a molecule signal is provided, and may comprise data collection means configured to collect signal data for at least two different molecules from a molecular identification or sequencing apparatus, the data including information corresponding to at least two signal parameters. The system may further comprise at least one processor having computer code operational thereon configured for determining the distribution of the frequency of occurrence of the values of each of the parameters. The processor may be further configured for creating a plurality of at least three-dimensional plots, wherein each plot comprises the determined values for a pair of parameters, such that, the determined values for each parameter is plotted versus each of the other remaining parameters. The processor may be further configured for determining the separation of values between different analyte molecules for each of the plots, and selecting at least one plot of the plurality of plots which includes a separation of values between the two analyte molecules greater than a predetermined amount. The processor may be further configured for determining the identity of signals according to their determined value location on the selected plot.

In some embodiments, a system of assigning a chemical identity to a molecule signal is provided, and may comprise at least one computer processor having computer code operational thereon configured for measuring a plurality of distributions of two or more signal parameters from signal data collected from a molecular identification or sequencing apparatus for known molecules. The computer processor may be further configured for determining at least one pair of parameters that best determine the separation of signals for identifying the known molecules. The computer processor may be further configured for using the pair of determined parameters, identifying one or more unknown molecules from second signal data collected from a molecular identification or sequencing apparatus.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Descriptions of well-known processing techniques, components, and equipment are omitted so as not to unnecessarily obscure the present methods and devices in unnecessary detail. Other objects, features and advantages of embodiments of the present disclosure will become apparent from the following detailed description. It should be understood, however, that the detailed description and the examples are provided for only some of the embodiments of the disclosure, and are given by way of illustration only, as various changes and modifications within the spirit and scope of the teachings of the subject disclosure will become apparent to those skilled in the art from this detailed description.

### BRIEF DESCRIPTION OF THE DRAWINGS

The following drawings form part of the present specification and are included to further demonstrate some of the embodiments of the present disclosure. Some embodiments may be better understood by reference to one or more of these drawings in combination with the detailed description of specific embodiments presented herein.

FIG. 1A illustrates 4(5)-(2-mercaptoethyl)-1H-imidazole-2-carboxamide adaptor molecules (hereafter referred to as “M”) showing how tautomerization presents different arrangements of hydrogen bond donors and acceptors.

FIG. 1B illustrates the adaptor molecule of FIG. 1A (left and right side) trapping dAMP (middle) via a network of hydrogen bonds (dotted white lines), according to some embodiments. In such embodiments, the sulfur atoms are bonded to gold electrodes, and current, I, is read as a bias, V, is applied across the tunnel gap. Individual 2D chemical structures were exported to Spartan′ 10 (Wavefunctions Inc.) to generate corresponding 3D structures that were energy minimized using the built-in MMFF molecular mechanics prior to the DFT calculation. The structures were first calculated using B3LYP with /6-31G* in vacuum (see structures for all four nucleotides are shown in FIG. 2). The gap size is set by the tunnel conductance and either maintained under servo control or left uncontrolled (but monitored via the baseline current).

FIG. 2 Binding motifs for all four bases (clockwise from upper left, A, C, T, G). The structures are calculated as described above with reference to FIG. 1.

FIGS. 3A-3C illustrate characteristics of signals generated by 1 mM phosphate buffer (pH=7) alone, according to some embodiments. FIG. 3A illustrates a trace showing peaks taken with the probe scanning at 2 nm/s, according to some embodiments, with the inset showing how the on-time is defined by the duration of a peak at half height. FIG. 3B illustrates a peak height distribution of pulses for (open circles) the probe scanning at 2 nm/s and (closed circles) a stationary probe, according to some embodiments. The distributions are normalized to 1 at the highest points and the total count rates are listed on the figure. FIG. 3C illustrates distributions of on-times for the spikes, according to some embodiments. These data were taken according to some embodiments, at a gap conductance of 12 pS, corresponding to a gap size of approximately 2.5 nm. The much larger number of counts in data taken with a stationary tunnel gap may reflect trapping of contamination in the gap.

FIGS. 4A-4E illustrate representative signals generated for the four nucleotides, dAMP (FIG. 4A), dCMP (FIG. 4B), dGMP (FIG. 4C), and dTMP (FIG. 4D), and d^{me}CMP (FIG. 4E) after removal of the water signal, according to some embodiments. Specifically, 10 μM nucleotide was dissolved in 1 mM phosphate buffer (pH=7) and the probe, functionalized with M scanned at 2 nm/s over an Au(III) surface also functionalized with M. The tunnel gap was set under servo control to a baseline current of 6 pA with a probe bias of 0.5V. The slew rate of the servo is much slower than the ms timescale of the pulses observed here. Approximate overall count rates are listed on the figure—which are much less than the pulse rates observed within the signal bursts shown here.

FIGS. 5A-5O illustrate amplitude distributions, (FIGS. 5A, 5D, 5G, 5J, 5M), on-time distributions (FIGS. 5B, 5E, 5H, 5K, 5N) and burst-frequency distributions (FIGS. 5C, 5F, 5L, 5I, 5O) for dAMP (first row), dCMP (second row), dGMP (third row), dTMP (fourth row) and dmeCMP (fifth row), generated according to some embodiments. Solid lines are fits to a log-normal distribution.

FIGS. 6A-6E illustrate “Clock-scans” over oligomers with the compositions listed, generated according to some embodiments. Oligomers were dissolved to a final concentration of 2 μM (intact oligomer) in 1 mM phosphate buffer (pH=7). Scan speeds are as listed on the figure. The burst time changes with the inverse of scan speed. Homopolymers always give regular bursts and alternating polymers always give alternating bursts when periodic signals are recorded.

FIGS. 7A-7C show some characteristics of the tunneling signals generated according to some embodiments. FIG. 7A illustrate properties of signal clusters and FIG. 7B illustrates pulse height and on-time, and FIG. 7C illustrates Pulse shape (quantified using Fourier and wavelet components).

FIG. 8 is a Fourier analysis of spikes generated according to some embodiments. In such embodiments, spikes are first baseline subtracted and amplitude-normalized, then inserted into a 4096 point data array, taken to be periodic for processing by an FFT. The power spectrum (going from 0 Hz to the Nyquist limit of 25 kHz) is separated into four equal bins that are each averaged to produce four coefficients.

FIG. 9 is an illustration of calculation of Haar wavelet components, according to some embodiments. For example, the first wavelet comes from convolution with 0, 1, −1, 0 to produce differences for all neighboring components. These differences may then be summed and averaged. The process is repeated for each successive (N^{th}) wavelet in which the filter is increased to 0, +2^{N} points, −2^{N} points, 0.

FIGS. 10A-10C illustrate an algorithm for locating a cluster, according to some embodiments. Each spike is replaced with a unit delta function centered at the middle of the spike (FIG. 10A) and a unit Gaussian of 4000 points FWHH placed on each spike (FIG. 10B). These Gaussians are summed and the cluster duration defined by the period over which this sum exceeds a threshold (FIG. 10C).

FIG. 11 illustrates an example of a Support Vector Machine (SVM) for a 2D space. The support vectors define the line that optimally divides the two data sets (open and filled squares). It is shown with a soft margin, tolerant of some mis-assignments and in non-linear form (the partitioning is not a straight line in this space). SVMs generalize this process to an (N+a)-dimensional space for N parameters with a being additional dimensions needed to deal with non-linearities in the data.

FIG. 12 shows a spike parameterization according to some embodiments. Each spike is characterized by a plurality of parameters (e.g., in some embodiments, up to about 30 parameters, for example).

FIG. 13 shows data pre-filtering according to some embodiments. For example, background signals may be removed by training a SVM with control data (no nucleotides). The parameter space used may be at least one of Spike Amplitude, Spike width; Spike Fourier Amplitude N, N=1 to 4 (for example), Spike phase, degrees, obtained as four numbers corresponding to the average of phase values in the four equally spaced frequency interval up to the Nyquist limit. Spike Wavelet Component N, N=1 to 9 (for example). In some embodiments, no cluster data may be used (since, in this example, clustered signals do not appear in these controls).

FIG. 14 shows SVM training according to some embodiments. The SVM may be trained on a randomly selected set of multiple spikes (e.g., 200 spikes) using data from each nucleotide. The accuracy with which the remainder of the set (in some embodiments, 400 data points) may be identified and recorded as a function of the parameter set used.

FIG. 15 shows the distribution of cumulative accuracies obtained from multiple combinations of parameters (e.g., 4,157 combinations of parameters), generated according to some embodiments. Most combinations yield 75% accuracy and a number yield about 80% (or better, including up to about 90-95%>) in calling each signal spike.

FIGS. 16A and 16B show the distribution of outcomes based on (FIG. 16A) individual spike characteristics generated according to some embodiments, and (FIG. 16B) cluster characteristics generated according to some embodiments. In some embodiments, the best outcomes based on spike characteristics like amplitude, spike on-time and width, call bases with about 50%> accuracy. Cluster parameters (spike frequency, cluster on time, number of peaks in a cluster) may produce accuracies of up to about 80%>.

FIG. 17 shows a 3D projection of a multiple (e.g., 14) parameter plot according to some embodiments, showing some part of the separation of the 5 bases and water into distinct clusters (overlapped somewhat in this 2D projection). With the exception of the G and water signals, data is spread out in distinct groups, which in some embodiments, suggests that discrete sets of configurations are sampled in the recognition tunneling gap. (Water signals were not removed from this data set by prefiltering.)

FIGS. 18A-18F illustrate current recordings (left) and spike height distributions for 10 μM dAMP in 1 mM phosphate buffer (pH 7.0) for (FIGS. 18A, 18B) bare electrodes, (FIGS. 18C, 18D) a bare probe and imidazole functionalized surface and (FIGS. 18E, 18F) and imidazole functionalized surface and thiophenol functionalized probe, according to some embodiments.

FIGS. 19A and 19B illustrate clock scanning, according to some embodiments; FIG. 19A illustrates voltages applied to the X and Y PZTs together with the recorded tunnel current showing bursts when the probe passes over a DNA oligomer. FIG. 19B illustrates the current distribution mapped onto the X,Y surface plane. One of skill in the art will appreciate that the signals tend to align along axes rotated by about 60°.

FIGS. 20A-20C illustrate periodic signal bursts from d(AAAAA) scanned at the speeds as marked (note that in FIG. 20C, four bursts are shown, so the distance per burst is about 0.3 nm for all three traces shown here).

FIGS. 21A-21F illustrate clock-scans over homopolymers as marked (FIGS. 21A, 21B, 21C) with spike height distributions to the right, and over heteropolymers as marked (FIGS. 21D, 21E, 21F). Spike height distributions may be bimodal over heteropolymers.

FIGS. 22A and 22B illustrate spike height distributions from homopolymers (FIG. 22A, d(AAAAA), FIG. 22B d(CCCCC)) shown as bars. The fitted distributions to the nucleotide data (FIGS. 5A-5O) are replotted here as solid lines.

FIGS. 23A and 23B illustrate FTIR of 4(5)-(2-mercaptoethyl)-1H-imidazole-2-carboxamide (FIG. 23A) in a monolayer; (FIG. 23B) in a powder. A gold substrate was cleaned by hydrogen flaming, and then immersed in a 0.2 mM ethanolic solution of 4(5)-(2-mercaptoethyl)-1H-imidazole-2-carboxamide for 24 h. The substrate was copiously washed with ethanol and dried by gently blowing a stream of dry nitrogen on the surface. Thickness of the monolayer was measured as 8.46±0.23 A by ellipsometry (the distance between 0 of the amide and S of the thiol is 8.44 A and the bond length of Au—S is about 2.45 A). The XPS data show that the monolayer contains C, N, O, and S atoms. FTIR spectrum of 4(5)-(2-mercaptoethyl)-1H-imidazole-2-carboxamide shows a similarity with that in a powder.

FIG. 24 illustrates distribution of the number of spikes in a cluster for dAMP scanned at 5 nm/s, according to some embodiments. The red line is a heavily damped log normal distribution centered on 13 spikes per cluster.

FIG. 25 illustrates peak bars are the distribution of calling accuracies based on various combinations of peak characteristics (including cluster information). Preferable parameter combinations yield a calling accuracy of a little over 80%. By voting within a cluster, either by simple majority (voted cluster bars) or adding probabilities returned by the SVM (added cluster bars) a significant number of parameter combinations yield >95% accuracy.

FIG. 26A is a graph representing recognition tunneling spectra, with respect to some embodiments of the present disclosure, with respect to a trace buffer.

FIGS. 26B-26H represent recognition tunneling spectra, with respect to some embodiments of the present disclosure, for (FIG. 26B) L-arginine, (FIG. 26C) glycine, (FIG. 26D) N-methyl glycine (sarcosine), (FIG. 26E) L-aspargine, (FIG. 26F) D-asparagine, (FIG. 26G) L-leucine, and (FIG. 26H) L-isoleucine. Insets on upper right show spike shapes (current scale 150 pA, timescale 20 ms). Data were taken at a tunneling set point of 4 pA at 0.5V using 100 μM solutions in 1 mM phosphate buffer.

FIGS. 27A-27C represent RT signals generated for GGG (FIG. 27A), GGGG (FIG. 27B) and GGLL (FIG. 27C), according to some embodiments.

FIG. 28 illustrates a plot of true positive rate vs. the number of spikes used for a majority vote, after scrambling spike order to remove correlations in clusters, according to some embodiments (Data is shown for odd N only to avoid voting ties).

FIG. 29 illustrates a plot of correlations generated between 40 parameters according to some embodiments (e.g., see Table 8 for which parameters are represented by the numbers). The values of the correlation coefficients are given by the scale on the right.

FIGS. 30A-30C illustrate how spike shapes discriminate data generated according to some embodiments. For example FIG. 30A illustrates a plot of spike width vs. average of first Fourier band for L- (blue) and D-aspargine (green). FIG. 30B illustrates a plot of average of the highest Fourier band vs. average of the lowest Fourier band for leucine (blue) and isoleucine (green). FIG. 30C illustrates a 3D plot of spike repetition rate vs. Fourier band 1 and the intensity at the middle of this band for glycine (blue) and sarcosine (green); in this case, the plotted data may group in distinct clusters, which, in some embodiments, reflect distinct binding geometries.

FIG. 31 illustrates a measured vs. actual ratios of L to (L+D) asparagine in mixed solutions generated according to some embodiments. The two sets of points at 0.5 and 0.75 on the vertical axis are repeated measurements. The fit passes through 0 and 1 and includes a quadratic component to take account of concentration-dependent association.

FIGS. 32 and 33 represent systems for at least one of conducting analysis and performing any of the methods taught by the present disclosure, including analysis of data using, for example, SVM methods and analysis and the like for at least one of removing background signals of raw data, qualifying and quantifying signal data, as well as including, in some embodiments, for comparing refined (and/or raw) signal data to stored signature signal data for any of the sequencing, detecting and/or otherwise identification of molecules (e.g., single molecules, chains of molecules, and the like).

FIG. 34A illustrates distributions of the values of a Fast Fourier transform (FFT) amplitudes for signal clusters in the frequency range of 22.6-23 kHz for methylglycine and leucine.

FIG. 34B illustrates shows the distribution of the values of FFT amplitudes for signal clusters in the frequency range of 8.6 to 9 kHz for methylglycine and leucine.

FIG. 34C is a histogram plot of the values from FIG. 34A (horizontal axis) and from FIG. 34B (vertical axis) for methylglycine (red spots) and leucine (green spots).

### DETAILED DESCRIPTION

Tunneling readout with metal electrodes requires small gaps (on the order of 0.8 nm) and the distribution of signals is very large (Tsutsui et al., 2010). In the present disclosure, an alternative referred to as recognition-tunneling is presented (Branton et al, 2008; Lindsay et al, 2010). In recognition tunneling, electrodes are functionalized with adaptor molecules, strongly-bonded to the metal electrodes at one end, and forming non-covalent bonds with target molecules at the other end. This permits much larger tunneling gaps (2.5 nm for the molecule described here, Chang et al, 2011) and reduces the signal distribution considerably (Chang et al, 2010). Using 4-mercaptobenzamide as the adaptor molecule, single bases embedded within a DNA oligomer may be identified, demonstrating the ability of recognition-tunneling to resolve single bases (Huang et al, 2010). In some embodiments, 4-mercaptobenzamide produced no signals from thymine, such that, a new adaptor molecule, 4(5)-(2-mercaptoethyl)-1H-imidazole-2-carboxamide was synthesized, the synthesis and characterization of which is described elsewhere (Liang et al, 2011). Signals are generated by all four bases as well as 5-methyl cytosine using this new molecule.

Theoretical simulations (Chang et al, 2009; Pathak et al., 2012) of currents in Recognition Tunneling have been carried out in “vacuum” at zero degrees Kelvin and they predict fixed current levels that signal the identify of a DNA base trapped in the junction in some fixed geometry. In reality, thermal fluctuations and the active intervention of water molecules generate a stochastic signal train (Lindsay et al., 2010; Chang et al, 2010; Huang et al, 2010; Chang et al, 2009). To a first approximation, the signal may be “random noise” and is has been shown (Huang et al, 2010; supplement) how random thermal motion, as sampled by an exponential matrix element, can generate signals that look a lot like those observed. Of course, truly random noise would be useless for sequencing, but diversity in the signals can be classified.

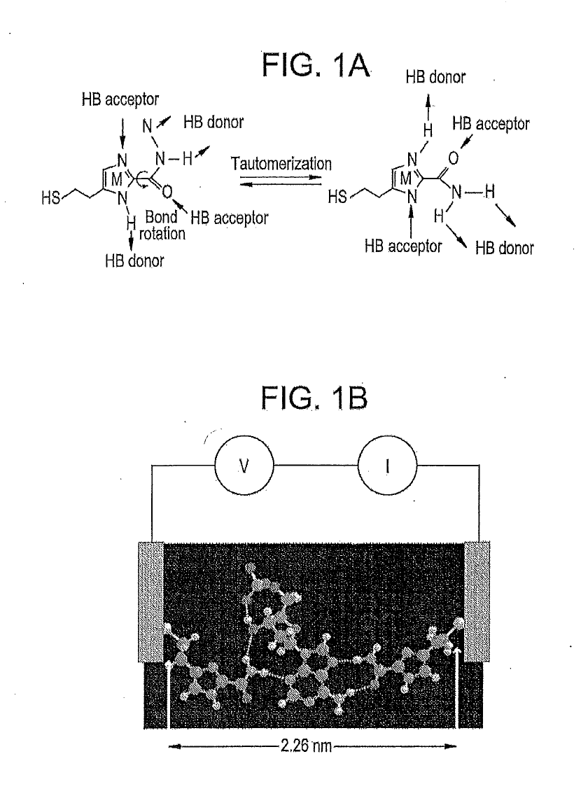

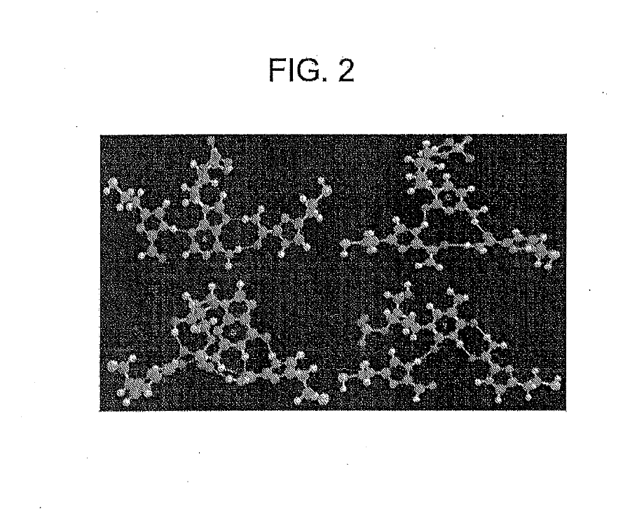

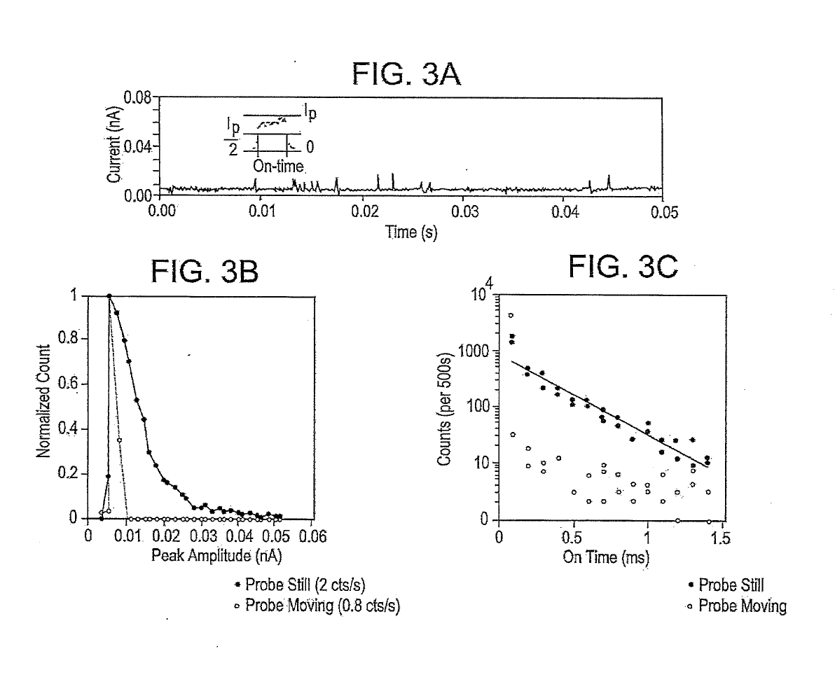

A certain fraction of the signals generated in a recognition tunneling junction are readily associated with a particular base. For example, as a tunneling probe is swept over an alternating DNA polymer comprising the repeated sequence motif (AT), the larger signal bursts {i.e., larger current peaks) are almost generated by C bases, and the smaller signal bursts generated by A bases. Nonetheless, the data considerably overlapped when a large number of reads are acquired. The may be illustrated, according to some embodiments, with raw data obtained with 4(5)-(2-mercaptoethyl)-1H-imidazole-2-carboxamide reader molecules. The layout of a tunnel junction for reading the identity of nucleotides or bases in a DNA polymer is shown in FIGS. 1A and 1B, which also shows one possible arrangement of the target adenosine monophosphate trapped in the junction. Arrangements for all four bases are shown in FIG. 2. 4(5)-(2-mercaptoethyl)-1H-imidazole-2-carboxamide generates a background signal even in the absence of a target base in the junction, and a typical trace of current vs. time is shown in FIG. 3A, which also summarizes the distribution of current-peak heights (FIG. 3B) and the width of the peaks at half height (“on-Time”, FIG. 3C). Thus, while 4(5)-(2-mercaptoethyl)-1H-imidazole-2-carboxamide gives reads for all four bases (and 5-methyl cytosine) a first issue is that a background signal should be discriminated against. Interesting, the characteristics of the signals change, depending upon whether the probe is moving (open circles in FIGS. 3B and 3C) or still (closed circles in FIGS. 3B and 3C). Thus rejection of this unwanted background is complicated.

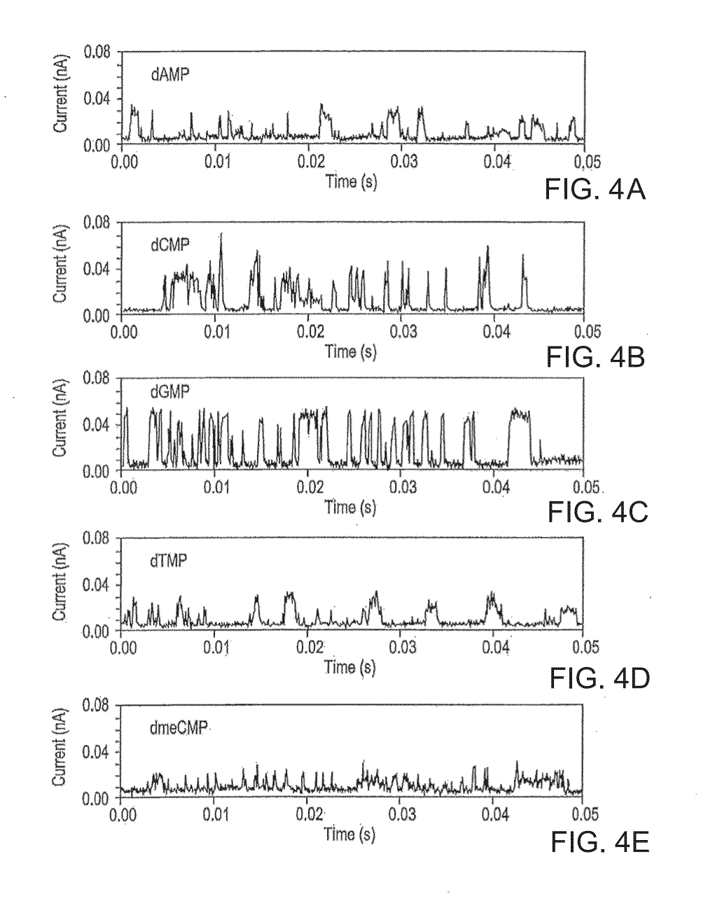

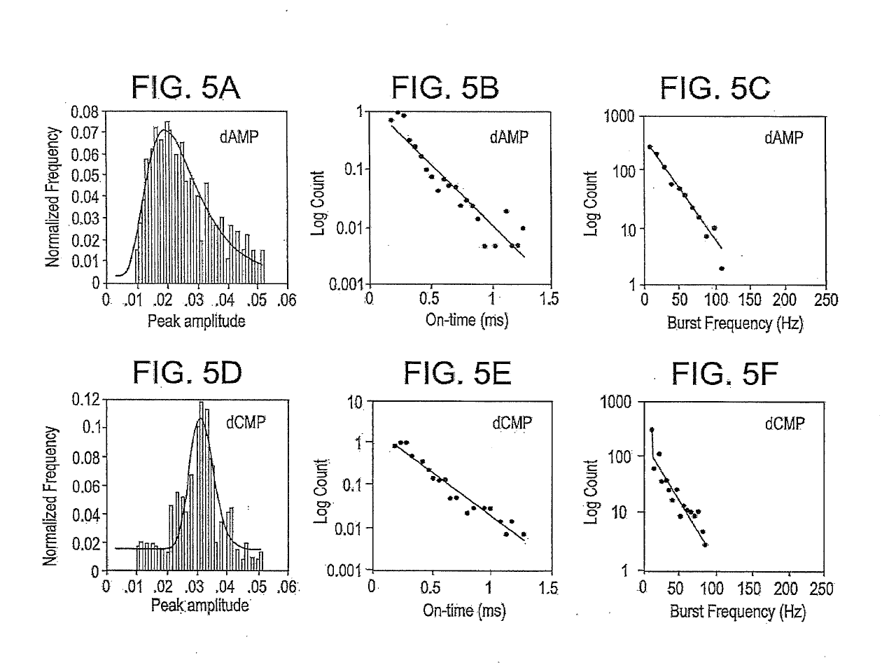

FIGS. 4A-4E show current vs. time traces for all 4 nucleotides and d(5-methylCMP) (hereafter dmeCMP). These are different from the signals generated by the aqueous buffer alone (FIG. 2) and, while signals like this are not observed in the absence of nucleotides, there are many “water-like” spikes in the signals obtained with nucleotides present. Examination of the nucleotide signals in FIGS. 5A-5O show that they look like classic “telegraph noise”—on/off signals with a rapid rise, a roughly flat top and a rapid fall. Accordingly, a “squareness” filter may be provided, in some embodiments, and configured as follows: (1) a share rise-time, the onset of a peak being marked by a first point within 3 pA above the average background, and a second point at least 8 pA above the first point (the first criterion eliminates peaks that do not start at the baseline). The time step between data points is 0.02 ms. (2) a rapid fall time with the data points on the falling edge following the same criteria in reverse. (3) a flat top to the signal; that is at least 10 data points before the fall with a variance such that the variance divided by the average of these high current points in less than 2 (this average is the peak current). Since the onset of the peak has to be between 8 and 11 pA (in some embodiments), this filter also rejects all peaks of less than about 10 pA in height. When applied to the raw data, almost all of the water signal is removed from the controls. However, a significant fraction of the nucleotide signal is taken out too (Table 1). This effect is extreme for 5-meC where over 90% of what is presumably nucleotide generated signal is removed by the filter.

Table 1 lists the signal frequencies defined as the total number of counts in an experimental run) divided by the duration of the run (10 s). The last two rows list the peak frequency and fraction of peaks passed by the “squareness” filter.

TABLE 1
Overall read frequencies with the probe scanning at 2 nm/s.

| | dAMP | dCMP | dGMP | dTMP | dmeCMP | Control |
|---|---|---|---|---|---|---|
| Avg. | 41.4 | 20.6 | 1.1* | 11.4 | 827.5 | 0.8 |
| peaks/s | | | | | | |
| Nucleotide | 0.86 | 0.72 | 100** | 0.50 | 0.99 | 0 |
| signal | | | | | | |
| fraction | | | | | | |
| Peaks/s | 5.98 | 2.23 | 0.97 | 4.45 | 27.77 | 0.015 |
| post- | | | | | | |
| Filtering | | | | | | |
| Fraction | 0.14 | 0.11 | 0.88 | 0.39 | 0.03 | 0.003 |
| passed | | | | | | |
| by filter | | | | | | |

*dGMP produces a lower count than the control alone, implying that the water signals are blocked by the presence of this nucleotide. The second row lists the fraction of signals due to nucleotides if the water signal were constant (**not true for dGMP).

Thus, a simple filtering of the data to remove the background signal rejects a lot of data that is generated by the target nucleotides. A more efficient filter is required.

Even after such filtering, the signals sometimes present challenges. FIGS. 5A-5O show a statistical summary of the pulses produced by each of the four nucleotides and dmeCMP. The first column gives the peak amplitude distributions. dCMP gives the largest peak amplitudes and dTMP gives the smallest while the dGMP, dAMP and dmeCMP distributions are largely overlapped. The solid lines are log-normal fits to the data (Eq. 1)

$$N ( i ) = {N}_{b} + \frac{{N}_{0}}{\sqrt{2 π w i}} exp ( \frac{- {ln ( \frac{i}{{i}_{p}} )}^{2}}{2 {w}^{2}} ) . \qquad ( 1 )$$

Here, N_{b} is a constant background, No a quantity that controls the height of the distribution, w a parameter that controls its width and iₚ is the peak current in the distribution. Peak currents obtained from these fits are listed in Table 2, showing how dCMP and dTMP are characterized by high and low currents respectively.

TABLE 2
Characteristics of the nucleotide signals for the probe scanning at 2 nm/s.

| Nucleotide | Iₚ | On-time (ms) | Burst f_{avg} (Hz) | Burst f_{1/e} (Hz) |
|---|---|---|---|---|
| damp | 0.023 ± 0.001 | 0.17 ± 0.03 | 32 ± 21 | 24 ± 1.6 |
| dCMP | 0.031 ± 0.0005 | 0.21 ± 0.03 | 25 ± 17 | 21 ± 6.6* |
| dGMP | 0.024 ± 0.003 | 0.27 ± 0.03 | 25 ± 22 | 39 ± 25 |
| dTMP | 0.013 ± 0.0003 | 0.44 ± 0.06 | 76 ± 59 | 101 ± 15 |
| dmeCMP | 0.021 ± 0.0005 | 0.19 ± 0.03 | 25 ± 16 | 11 ± 1.2 |

*Fits to the burst frequency distribution were single exponentials with the exception of data for dCMP that included a second slow component.

A second obvious characteristic lies in the “on-time” for each pulse. Inspection of FIGS. 5A-5O shows that dTMP appears to produce longer pulses. The distributions of on-times are given in the second column of FIGS. 5A-5O and they are fitted by exponentials,

$$N ( {t}_{on} ) = A exp ( \frac{- {t}_{on}}{{t}_{1 \text{/} e}} )$$

as would be expected for a Poisson process (solid lines on the figures). Values for t_{1/e} are listed in Table 2 also. dTMP signals may be distinguished by longer on-times.

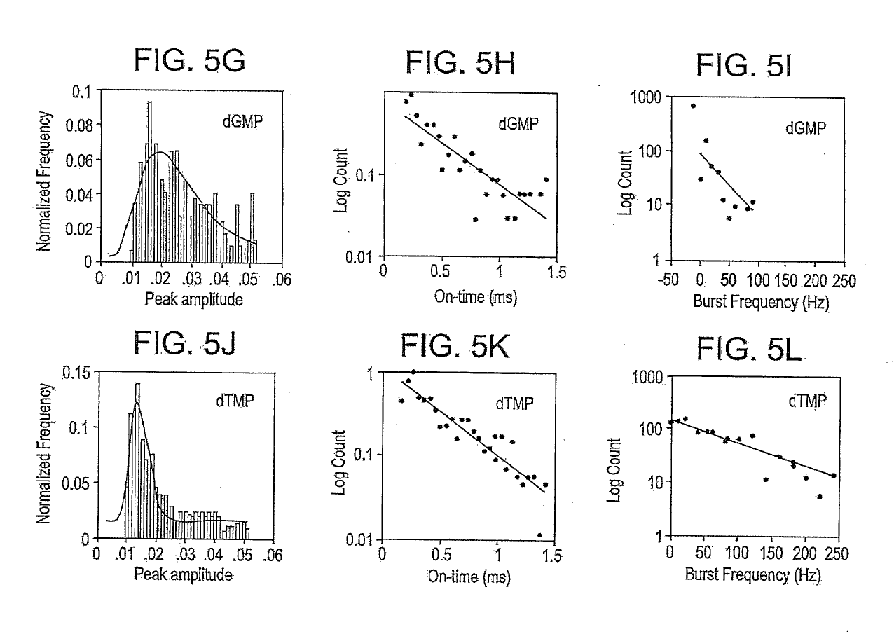

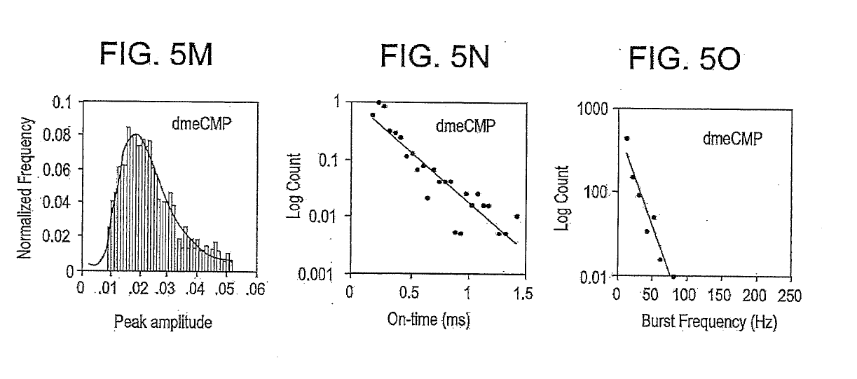

Another parameter is the frequency of signal spikes in a cluster (FIGS. 6A-6E, and FIG. 7). These data clusters may be defined operationally by a sliding average over the data stream. When a peak is detected, the number of other peaks within 2000 data points each side (40 ms each side) is counted and a frequency calculated. The frequency is recalculated for each point in the data in turn, and the resulting distribution of frequencies recalculated. Isolated peaks (more than ±40 ms from a neighbor) produce values of zero. The averages for each nucleotide are listed in Table 2. The frequencies themselves are exponentially distributed (third row FIG. 4C) according to

$$N ( f ) = A exp ( \frac{- f}{{f}_{1 \text{/} e}} )$$

and the corresponding values of file are listed in the last column of Table 2. dGMP and dTMP are characterized by high burst frequencies.

Thus, it appears that C, T and G can be distinguished from A and meC. However A and meC in this data set (with much of the meC data removed) are not easily separated. A similar type of analysis was carried out for DNA bases read with a benzamide molecule (Huang et al, 2010). In that work, it was demonstrated how a combination of both signal height and signal frequency could be used to improve accuracy with which bases could be called using these stochastic signals. Nonetheless, the assignment is often made with a small probability of being correct, owing to the very broad distribution of characteristics of the signals (as shown in FIGS. 5A-5O).

Even without the adaptor molecules that interface the target molecules to the metal electrodes, tunneling measurements can give signals that are somewhat representative of the chemical identity of trapped molecules, as shown in the recent work of the Kawai group (Tsutsui et al, 2010; Tsutsui et al, 2011). However, the measured current distributions are even broader so the probability of correct based-call on a single read is even smaller than is the case with the recognition-tunneling.

Recognition-tunneling may also be used to recognized amino acids, as taught in PCT Publication No. WO/2013/116509 (claiming benefit of U.S. Provisional Application Ser. No. 61/593,552, filed on Feb. 1, 2012), both disclosures of which are hereby incorporated by reference. While distinct signals are obtained, it may be challenging because of the need to identify 20 amino acids (as opposed to 5 types of DNA base and the background water signal).

FIGS. 6A-6E show trains of signals generated as a scanning probe is moved over DNA molecules {(see FIGS. 7A-7C for a definition of the signal characteristics). The signals come in distinct bursts of duration Tb. When Tb is plotted versus 1/(scanning speed), the result is a straight line with a slope of 0.3 nm, corresponding to the distance over which the adaptor molecule on the probe remains bound to a DNA base. The properties of these bursts are illustrated schematically in FIG. 7A. Taking signal spikes to be any set of data points that rises above the average baseline current (lb) by more than 1.5 times the variance of the baseline current (σ), a typical signal comprises of a burst of spikes that lasts for a period Tb, dictated entirely by the probe speed. The different bases produce signals within a burst at different rates, fₛ. The signals are stochastic, so fₛ is not a constant frequency, but is defined by the number of spikes in a burst divided by the duration of a burst. This number does not depend on scan speed. Another characteristic of the burst is the distribution of times (T_{off}) between pulses. While this is related to fₛ it is a distinct quantity in that it depends on the width of the spikes also.

Each spike itself is characterized by several parameters. One is the average peak current, Iₚ, above the baseline current, ¾(see FIG. 7B). This is defined by the average of all the data points within a “flat top” part of the signal. This flat top is, in turn, defined by all the points near the highest point of the signal (Iₘₐₓ) such that (Iₘₐₓ−I)/I_{P}≦2. Another is the full width of the spike at I_{P}/2, shown in the figure as Tₒₙ.

The intrinsic shape of the spike is significant, as can be seen by inspecting the raw data as shown in FIGS. 4A-4E. Some representative peaks pulled from each of these traces (and normalized in height) are shown in FIG. 7C. These properties are referred to as spike parameters.

In addition to the intrinsic properties of each spike, the context of the spikes may be important in some embodiments. For example, signals occur in bursts, and it has been demonstrated elsewhere that each burst is generated by a single base trapped in the tunneling junction (Huang et al., 2010). The intrinsic duration of the signal (with no force applied to pull the molecule through the tunnel junction) is about 3 s. When a probe is moved over the target, the duration of each burst is given approximately by

$${T}_{b} = \frac{d}{V} .$$

where d is about the size of a base (0.3 nm) and V is the tip speed in nm/s. For the examples analyzed here, V was 2 nm/s so the burst durations were typically 0.15 s. Properties of the bursts are referred to as cluster characteristics.

Parameters used in assigning the chemical origin of each peak in, according to some embodiments, include:

### Spike Parameters:

Spike Amplitude (pA)
Spike width (0.02 ms samples)
Spike Fourier Amplitude N, N=1 to 4
Spike phase, degrees
Spike Wavelet Component N, N=1 to 9

### Cluster Parameters:

Number of Peaks In a Cluster
Cluster on Time (%)
Spike Frequency (spikes within ±2000 0.02 ms samples)
Cluster frequency N, N=1 to 4
Cluster phase component N, degrees

Spike Amplitude. This is the average peak amplitude (in picoamps) as defined above.

Spike Width. This is the full width of the peak at half the average peak height (analyzed here in terms of the number of 0.02 ms sample points).

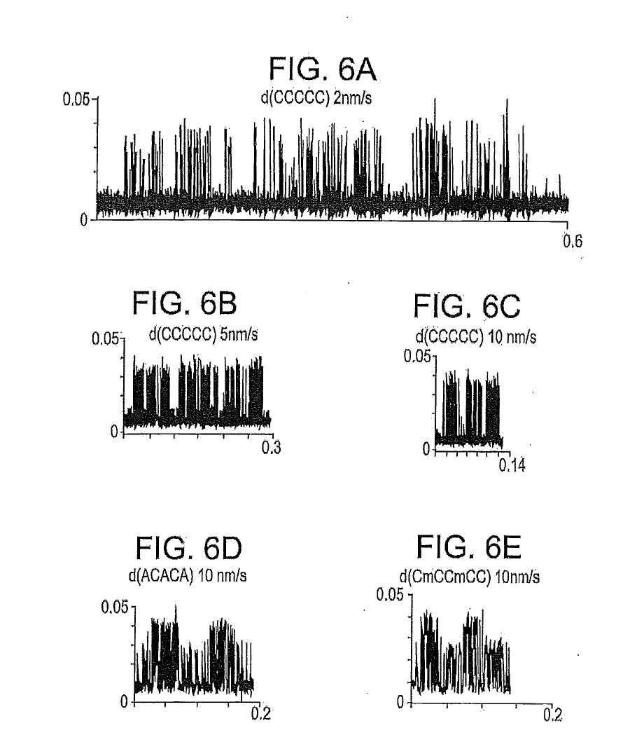

Spike Fourier Component N. Each spike is embedded into a data array of a fixed length and the power spectrum (\sqrt{Re²+Im²}) obtained (by FFT) out to the Nyquist limit. This frequency interval is divided into 4 bins and the average value of the power density in each bin (N=1 to 4) is recorded. The process for obtaining Fourier components is illustrated in FIG. 8.

Spike Phase Component N. The FFT also produces a phase, 0, that can be averaged over the four frequency intervals, obtained from

$$Ø = {tan}^{- 1} ( \frac{Im}{Re} )$$

where Im is the imaginary value of the FFT and Re the real part. The average is calculated from all of the phase values in each of the four frequency blocks between zero and the Nyquist limit.

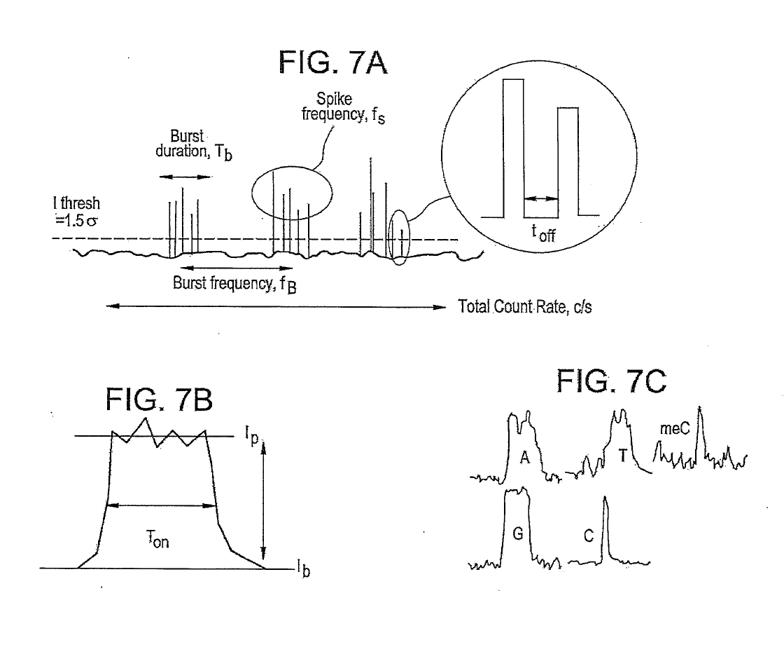

Spike Wavelet Component N. This is the Nth component (N=1 to 9) of a decomposition of the spike into Haar wavelet components as illustrated in FIG. 9 (for a description of the Haar Wavelet see Matlab Toolbox, available on the world wide web at matlab.izmiran.ru/help/toolbox/wavelet/ch06a32.html). The whole dataset has the background removed, then is processed by the Haar wavelets. At the location of each peak, the wavelet coefficients are extracted and averaged for the duration of the peak. The first wavelet component is obtained by applying the Haar transform to each point to generate a series of 4096/2 differences, Δ(1)_{B}=I₂ₙ₋₁−I_{2η}. These differences are squared, summed and divided by to produce an average value for Wavelet(1). At higher levels, N>1, the Nth wavelet component is produced using the average of M_{N}=2^{N} consecutive points,

$${\overline{I ( N )}}_{m} = \frac{1}{{M}_{N}} {\sum }_{i = 1}^{{M}_{N}} {I}_{{mM}_{N} + i - 1}$$

to produce the differences,

$${Δ ( N )}_{n} = {\overline{I ( N )}}_{2 n - 1} - {\overline{I ( N )}}_{2 B} .$$

The Wavelet(N) is then calculated by averaging these difference values. Given the limited time response of the current recording system, only the larger wavelet components are useful.

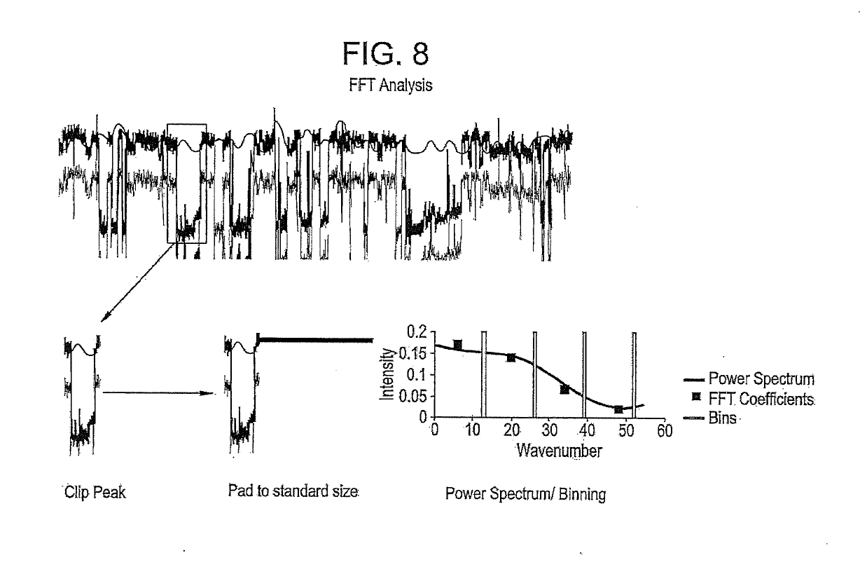

Number of Peaks In a Cluster. Clusters are defined operationally using the algorithm illustrated in FIGS. 10A-10C. The location of the center of each peak is identified with a 1 in an otherwise null array (FIG. 10A). Each point is then convolved with a Gaussian of unit height and a full width at half height of 4000 0.02 ms sample points (FIG. 10B). The Gaussians are summed (FIG. 10C) and the boundaries of a cluster defined by the points at which this sum falls below 0.1 (“Threshold” on FIG. 10C). This point may be somewhat arbitrary, but values in this range (0.01 to 0.25) work well (according to some embodiments). Once clusters are identified, the number of peaks in a cluster is a parameter assigned to each peak in that cluster.

Cluster on time. This is the ratio of the sum of the full widths of all peaks in a cluster to the total duration of the cluster, expressed as a percentage in the code used here. Each peak in a cluster is assigned the value calculated for the cluster.

Spike Frequency. This is calculated independent of the cluster definition and is the number of peaks found within ±2000 0.02 ms sample points of the center of a given peak. The value is assigned to the peak about which the value was calculated. The calculation is carried out in the following way: Each spike is represented by a 1 at its center location. A Gaussian of unit height and 4000 points full—width at half—height is centered at each 1 in the array. For each spike location, all the Gaussians in the array are summed according to their value at that point, generating a number that reflects the spike frequency in the neighborhood of each spike.

Cluster Frequency. N Each cluster is loaded into an array of 4096 points and the FFT calculated for the entire cluster as described above for spikes. It is resolved into nine bins covering the frequency range up to the Nyquist limit.

Cluster Phase N. This is calculated analogously to spike phase, but for the whole cluster. This parameter set was not used in the analysis discussed here.

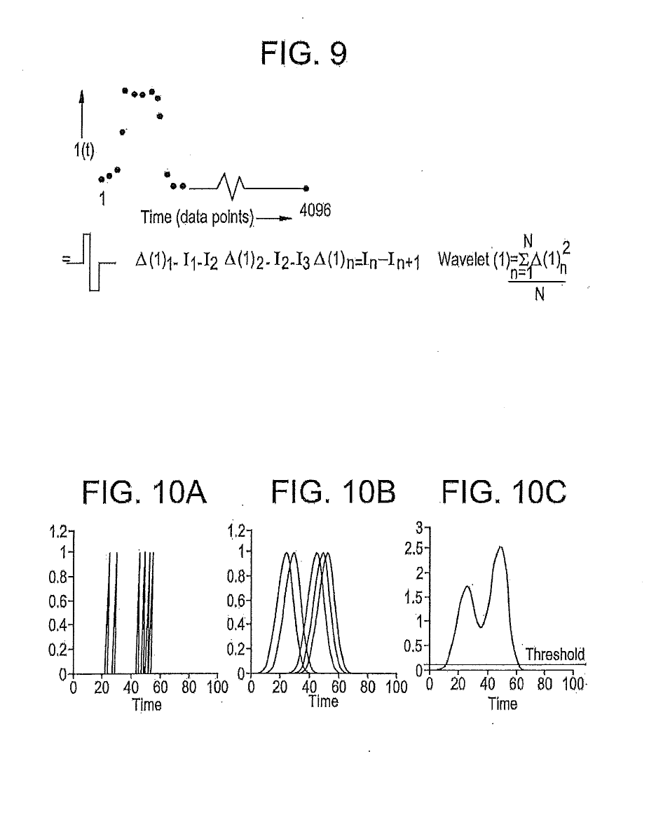

This set of 30 parameters, listed for each spike, constitutes a potential basis for assigning the chemical origin of each spike. Thus each spike can be represented as a point in a space of up to 30 dimensions. An issue with respect to assigning signals is determining how best to divide this space using a training set of data. Many procedures are available for doing this of which one of the beast known is the Support Vector Machine (as previously identified, also referred to as SVM), illustrated in FIG. 11. Some embodiments of the present disclosure used a routine published by Chih-Chung Chang and Chih-Jen Lin (LIBSVM: a library for support vector machines. ACM Transactions on Intelligent Systems and Technology, 2:27: 1-27:27, 2011. Software available on the world wide web at csie.ntu.edu.tw/˜cjlin/libsvm). Version 3.11 was used in the present work.

The library comes with a number of adjustable parameters that require setting in a manner appropriate to the issue at hand. These settings are listed in Tables 3 and 4 which summarize the accuracies that result from various parameter combinations. They are referred to as Easy, Scaled and Unsealed, defined as follows:

Easy: Easy.py is a predefined python script that is distributed with LIBSVM to automatically determine a few of the adjustable parameters of the SVM. The script iteratively searches the SVM parameters (gamma, C) to specify the most accurate kernel.

Scaled: Before training, both the training and testing datasets are scaled so all the parameters range from −1 to 1. This helps to prevent one parameter from overwhelming the SVM data.

Unsealed: The SVM is trained with data that has not been scaled.

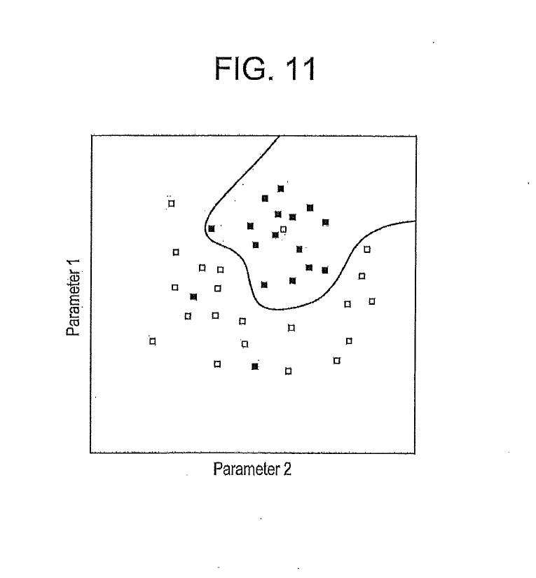

The first step may comprise running data sets taken with each of the 4 nucleotides, d(methylCMP) and the control (buffer with no nucleotides) through a routine that compiles a list of the thirty parameters for each spike in the data set (FIG. 12).

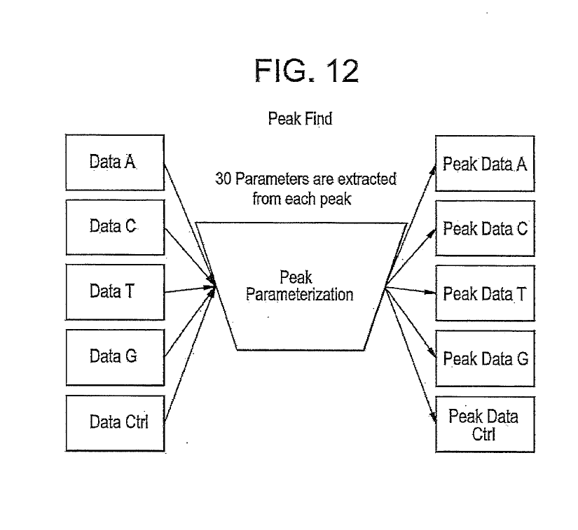

The second step may comprise of filtering out the water (control) spikes (FIG. 13). This is done by training the SVM with the control data alone using just the spike parameters as the water background contains no clusters. These are amplitude, spike width, all spike frequencies and phases. The trained SVM is then used to flag all water-generated peaks for removal, producing five sets of filtered data.

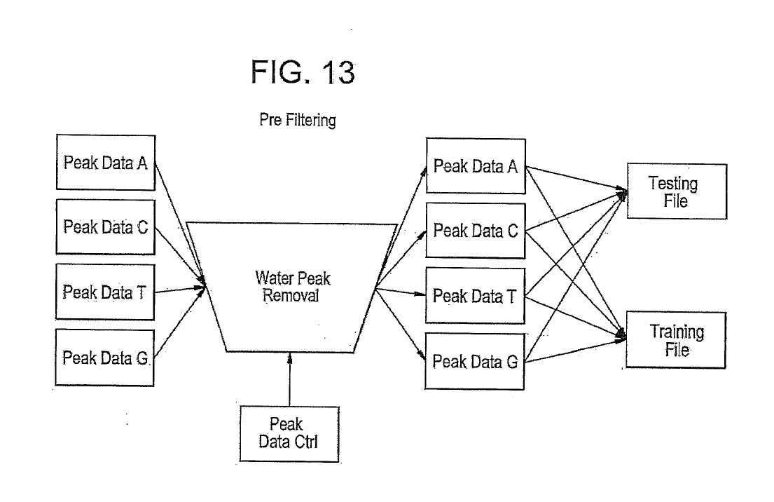

The importance of various combinations of the parameters listed above by training the SVM using a randomly selected subset of a plurality of spikes (e.g., in some embodiments about 200 spikes) from the water filtered data sets (FIG. 14) was investigated. A support vector is generated for each nucleotide, and the SVM then fed the entire data set for each of the four nucleotides and 5-methyl C) and an accuracy determined. The accuracies listed here are cumulative: that is to say the fraction in error is the sum of all the miscalled bases divided by the total number of spikes in all the data sets.

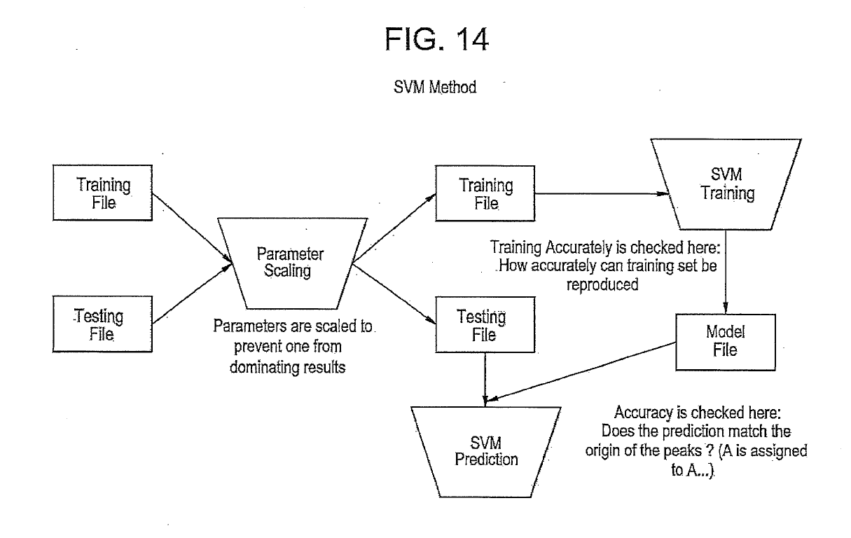

Remarkably, many combinations of parameters yield high accuracy calls for each single spike in the data set. FIG. 15 shows the distribution of cumulative accuracies for a total of 4,157 combinations of parameters tested. Most combinations produce an accuracy of 75% or better assignment of each single base from among the data set for all 5 bases (i.e., four bases plus 5-methyl C). The top nine combinations, each of which yielded 80% or better (up to 90, 95, or 99%) base calling are listed in Table 3. Interestingly, the best combination (84% accuracy) used only four parameters, each of which was a cluster parameter:
ClusterOnTime (%) clusterfreq3 clusterfreq8 clusterfreq9

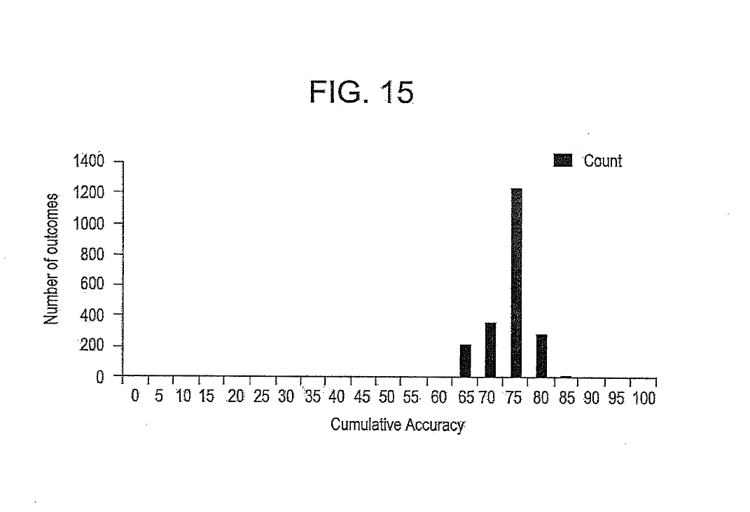

Each of the top nine combinations (Table 3) include cluster parameters. Indeed, all of the more accurate base-calling combinations include cluster data, as illustrated by the distributions in FIGS. 16A and 16B.

TABLE 3
The top nine parameter combinations together with the SVM setting.

| Cumulative | SVM | |
|---|---|---|
| Accuracy | Setting | Parameters Used |
| 84 | Unscaled | ClusterOnTime(%)clusterfreq3 clusterfreq8 clusterfreq9 |
| 80.8 | Unscaled | Spike Amplitude (pA) NumPeaksInCluster ClusterOnTime(%) |
| | | freq3 clusterfreq1 clusterfreq2 clusterfreq3 clusterfreq5 |
| | | clusterfreq6 clusterfreq7 clusterfreq8 wavelet5 wavelet7 |
| 80.6667 | Easy | NumPeaksInCluster clusterfreq1 clusterfreq4 clusterfreq5 |
| | | clusterfreq7 wavelet 1wavelet3 wavelet7 |
| 80.6667 | Unscaled | Spike Amplitude (pA) Spike Frequency (spikes per 4000 |
| | | samples) NumPeaksInCluster ClusterOnTime(%) clusterfreq2 |
| | | clusterfreq3 clusterfreq4 clusterfreq5 clusterfreq7 wavelet2 |
| | | wavelet4 wavelet7 wavelet9 |
| 80.5333 | Unscaled | NumPeaksInCluster ClusterOnTime(%) freq2 clusterfreq2 |
| | | clusterfreq7 clusterfreq8 wavwlet2 |
| 80.5333 | Easy | Spike Amplitude (pA) NumPeaksInCluster ClusterOnTime(%) |
| | | freq3 clusterfreq1 clusterfreq2 clusterfreq3 clusterfreq5 |
| | | clusterfreq6 clusterfreq7 clusterfreq8 wavelet5 wavelet7 |
| 80.2667 | Unscaled | Spike Amplitude (pA) NumPeaksInCluster ClusterOnTime(%) |
| | | freq1 freq4 clusterfreq1 clusterfreq2 clusterfreq5 clusterfreq7 |
| | | clusterfreq8 clusterfreq9 wavelet2 wavelet3 wavelet5 |
| 80.1333 | Easy | Spike Width (Samples) ClusterOnTime(%) freq1 freq2 freq4 |
| | | freq7 clusterfreq2 clusterfreq4 clusterfreq5 clusterfreq6 |
| | | clusterfreq7 wavelet1 wavelet2 wavelet3 wavelet4 wavelet6 |

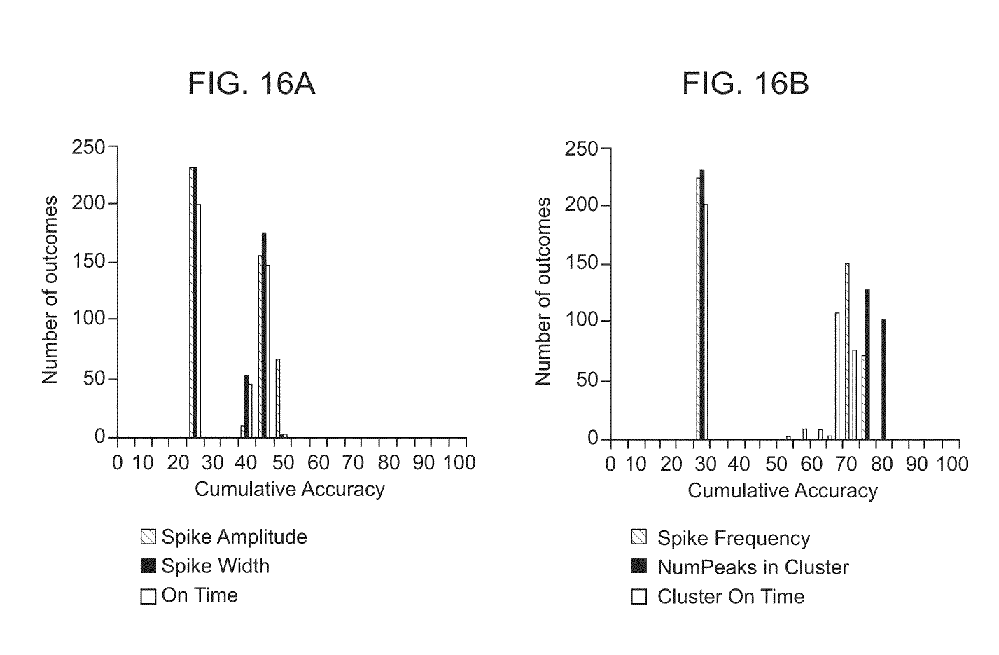

A display of the separation of data that is achieved by selecting a 2D projection of a 3D plot is presented in FIG. 17, of an example of 12D data (projected onto 3 axes as follows: Vector X: Spike Freq, Cluster Length, Freq 5, Cluster Freq 3, Cluster Freq 8; Vector Y: Cluster on Time, Freq1, 2, 4,6 ClusterFreq 5, ClusterFreq 9; Vector Z; ClusterFreq 1, ClusterFreq 4, ClusterFreq 7). A 2D view was then chosen such that much of the separation can be visualized. It is interesting to note that much of the data clumps into distinct groups, suggesting that there are a discrete set of configurations for molecules in the tunnel gap. The data are separated well at the 80% level, but multiple views are required to show this. Nonetheless, even in this 2D projection, the data is separated. Inspection of many such plots (using axes that separate the data) show the following common characteristics:

(a) Data for A, C and T are widely spread.
(b) These data tend to form multiple clusters, suggesting that there are several distinct binding motifs responsible for the signal.
(c) Data for G and water tend to be localized.
(d) Data for 5-methylC tend to be surrounded by A data points, recapitulating the similarities observed in the simple analysis of peak characteristics (FIGS. 5A-5O).

Each of the top nine combinations (Table 3) include cluster parameters. Indeed, all of the more accurate base-calling combinations include cluster data, as illustrated by the distributions in FIGS. 16A-16B.

Thus far, the analysis has been restricted to the one data set taken with a moving probe (2 nm/s) and servo control on. However, in some embodiments, the top parameter combinations are robust against even changes in the experimental protocol. To show this, three duplicate data sets in three different conditions were collected:

Set 1: Probe scanned at 2 nm/s, tunnel gap maintained under servo control
Set 2: Probe scanned at 2 nm/s, no servo control
Set 3: Probe stationary, tunnel gap maintained under servo control

It was understood that the servo-control may cause some distortion of the longer pulses, while operation without servo control (set 2) contaminates the data with noise from events where the probe crashes into the surface. The stationary gap accumulated contamination and gave a very high count rate even in the control experiments (no nucleotides added) so the “water filtering” removed most of the spikes accumulated in the data set (but leaving a residue comparable to the count rates in the uncontaminated experiments). The SVM was trained with a random selection of known spikes from all three data sets, and the accuracies tested using pooled data from all three trials. Remarkably, the top combinations again produced nearly 80% accuracy (Table 4) even though only one set of Support Vectors was used for all three data sets (containing a total of 21,000 signal spikes). Thus, even though each experimental approach was somewhat different (the stationary probe produced much more water background and the servo-off runs contained noise from the occasional probe crash) the same set of support vectors could be used to call data from all three experiments. The accuracies listed for the top parameter combinations in Table 4 are for calling bases from data pooled from all three experiments and it can be seen that the accuracies are only a little smaller than those obtained from analyzing a single type of data (as presented in Table 3).

TABLE 4
Cumulative accuracies for three data sets obtained in three different
experimental conditions using one set of support vectors.

| Cumu- | | |
|---|---|---|
| lative | | |
| Accu- | SMV | |
| racy | Setting | Parameters Used |
| 79.6 | Unscaled | ClusterOnTime(%) clusterfreq1 clusterfreq6 |
| 78.9 | Unscaled | ClusterOnTime(%) freq2 freq3, freq4 clusterfreq6 |
| | | clusterfreq5 clusterfreq6 clusterfreq7 clusterfreq9 |
| | | wavelet5 |
| 78.4 | Unscaled | Clusterfreq1 clusterfreq3 clusterfreq4 clusterfreq8 |
| | | clusterfreq9 wavelet8 |
| 77.9 | Unscaled | Spike Amplitude (pA) NumPeaksInCluster freq2 freq5 |
| | | clusterfreq1 clusterfreq5 clusterfreq8 wavelet4 |
| | | wavelet5 |
| 76.9 | Unscaled | Clusterfreq1 clusterfreq2 clusterfreq8 clusterfreq9 |
| 76.5 | Unscaled | Clusterfreq2 clusterfreq7 |
| 76.4 | Unscaled | Spike Amplitude (pA) NumPeaksInCluster freq1 freq3 |
| | | clusterfreq2 clusterfreq3 clusterfreq4 wavelet4 |
| | | wavelet5 |

Only one set of Support Vectors was used for all three data sets.

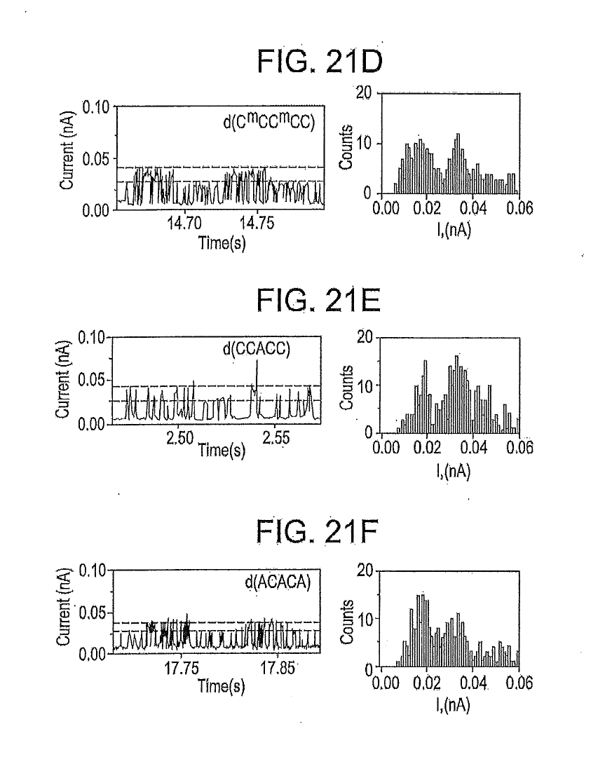

As pointed out earlier, much of the data may comprise repeated reads on the same base. The distribution of the number of spikes in a cluster follows a heavily damped log-normal distribution. An example of such a distribution (for dAMP with the probe scanned at 5 nm/s) is given in FIG. 24. Most of the data contains two or more spikes with clusters of up to 13 spikes being common. It will be recognized that the accuracy of calls can be further improved by using all the spikes in a cluster that is identified as coming from a single base. That is to say if:

(a) A cluster length (in time) corresponds to a base dimension (in space, i.e., 0.3 nm) given the known speed with which the molecules pass the tunnel gap and (b) all calls within that cluster assign the same base, then the occurrence of repetitive, sequential calls can be used as an additional factor in calling bases. This latter check on calling accuracy is important, because the SVM does not reject data points, so data for which it is untrained will be miscalled.

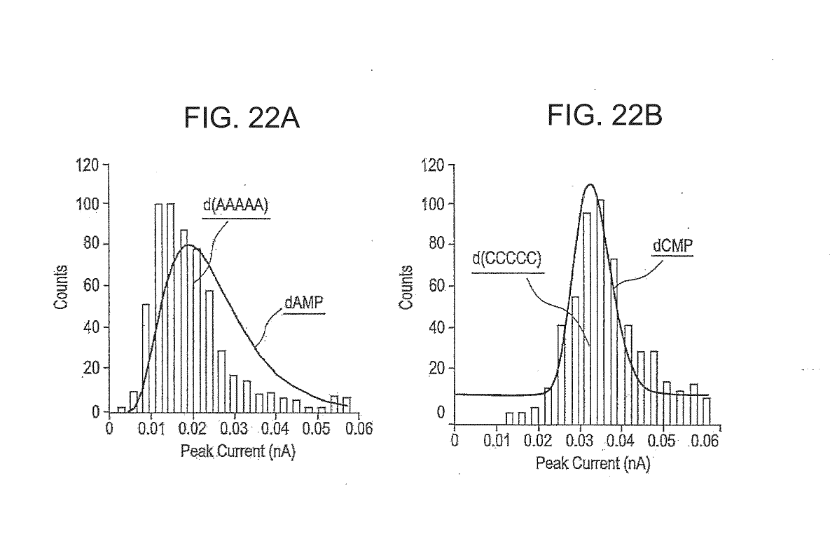

In some embodiments, the SVM code was configured to report probabilities for the call for each base and then tabulated these along with the data generated for each spike. As expected, spikes within the same cluster were often called as the same base and this repeated data may be used to enhance the accuracy of the calls. In one case, votes counted within a cluster calling the base by the majority vote. Thus an AACAC read within a cluster is called an A. In some embodiments, the probabilities reported by the SVM code were used, adding each probability and calling the winner from the largest sum (this differs from the vote in biasing the call towards assignments made with the larger probabilities). In both cases, the accuracy, determined by comparison with the frequency of correct calls given the known identity of the target moved up to >95% compared to −80% that was obtained without the use of cluster voting algorithms (FIG. 25). Some calls exceed 99% accuracy (as reported by the SVM). Examples of calls with associated probabilities as a function of cluster size are given in Table 5. The first column lists the number of spikes in a cluster while subsequent columns list the accuracy of the call as returned by the SVM as dAMP, dCMP, dGMP, dTMP and dmeCMP (the target in this case was dAMP). Some of the clusters with 10 spikes in them can be called to better than 99% accuracy.

TABLE 5
Examples of base calling accuracy from a selection of different clusters of
various size for a sample of dAMP.

| Spikes | | | | | |
|---|---|---|---|---|---|
| in | | | | | |
| Cluster | P(A) | P(C) | P(G) | P(T) | P(meC) |
| 3.0000 | 0.67495 | 0.11754 | 0.13033 | 0.010480 | 0.066702 |
| 10.000 | 0.64892 | 4.8600e-06 | 5.2500e-05 | 3.6500e-06 | 0.35102 |
| 10.000 | 0.91724 | 0.0084734 | 0.0051912 | 0.052177 | 0.016914 |
| 10.000 | 0.30575 | 0.15436 | 0.075989 | 0.16042 | 0.30348 |
| 10.000 | 0.99999 | 5.0600e-08 | 4.4600e-07 | 1.1600e-05 | 7.0100e-07 |
| 10.000 | 0.99971 | 1.6800e-07 | 0.00012862 | 0.00013496 | 2.1500e-05 |
| 10.000 | 0.99852 | 9.5000e-06 | 0.0014556 | 3.2200e-06 | 1.3000e-05 |
| 10.000 | 0.94007 | 6.4500e-06 | 7.6000e-05 | 0.00012719 | 0.059722 |

### Spikes in P(A) P(C) P(G) P(T) P(meC) Cluster

10.000 0.65244 2.2400e-06 0.00049840 1.7000e-06 0.34706
1.0000 0.61618 0.054975 0.11677 0.027437 0.18463
2.0000 0.97333 0.00014965 0.0099610 0.00020670 0.016358
2.0000 0.28232 0.28171 0.068545 0.090825 0.27659
1.0000 0.48941 0.17596 0.23492 0.025562 0.074144
1.0000 0.87122 0.046533 0.063004 0.0062350 0.013007

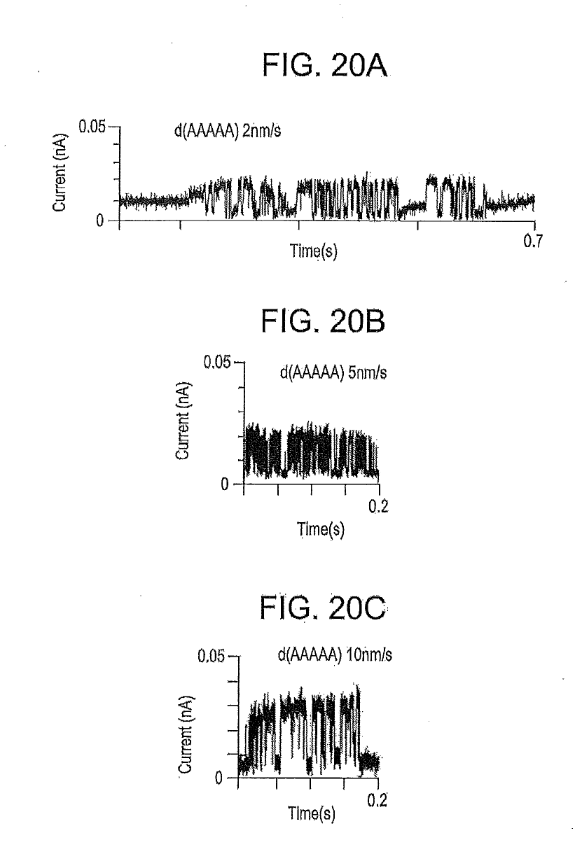

In some embodiments, the SVM may suffer from a drawback—when presented with new types of signal, it calls the new points as one of the bases it was trained on, regardless of how far they lie from the training data, according to the support vectors they lie behind. Thus, while blind trials with a single nucleotide support the 80% base calling accuracy, data obtained with mixtures of nucleotides are much less accurate (failing extremely in some cases—for example, an equimolar mix of dAMP, dTMP and dGMP was analyzed has having no T's). In such embodiments, a source of the issue may be inter-nucleotide interactions in the tunnel junction, with hydrogen bonds between nucleotides replacing interactions with water molecules and the adaptor molecules. In such a case, then these interactions probably also occur when only a single type of nucleotide is used. Since inter-nucleotide interactions may be more limited when the bases are incorporated into a DNA oligomer, this may account for the differences between the distributions measured for nucleotides and for the corresponding DNA oligomers (FIGS. 22A and 22B). A DNA sequencing device may be better trained on homopolymers than with nucleotides.

In summary, 4(5)-(2-mercaptoethyl)-1H-imidazole-2-carboxamide, in some embodiments, generates relatively large recognition-tunneling signals, despite incorporating an additional two methylene groups in the linker to the electrode. This demonstrates how the electronic states of the adaptor molecule may be engineered to increase the level of tunneling signals. Signals may be obtained from all four bases and 5-methylC, though the distributions of peak amplitudes are overlapped significantly. Nonetheless, the signals are distinctive such that trains of signal bursts may be recognized when a tunneling probe is scanned over DNA oligomers. The burst time is inversely proportional to the probe speed and corresponds to a spatial distance of 0.3 nm (i.e., about the size of a base). These scanning data can be used to set limits on the on- and off-rates for the complex of adaptor molecules with the targets. The off-rates are slow (corresponding to lifetimes of seconds) consistent with AFM measurements of the lifetimes of hydrogen-bonded complexes in a nanogap (Fuhrman et al., 2011; Huang et al., 2010). This behavior has recently been explained as a consequence of the bond confinement in the gap (Friddle et al., 2008). The on-rates are fast, probably too fast to be measured with the techniques used here, but certainly consistent with DNA sequencing speeds of many tens of bases per second.

The wide distributions of measured parameters are inconsistent with base calling from single molecule reads, but a multi-parameter analysis shows that most signal spikes contain chemical information if analyzed appropriately. This analysis suggests a wide range of binding motifs in the tunnel gap and also points to complications owing to internucleotide interactions when free nucleotides are used.

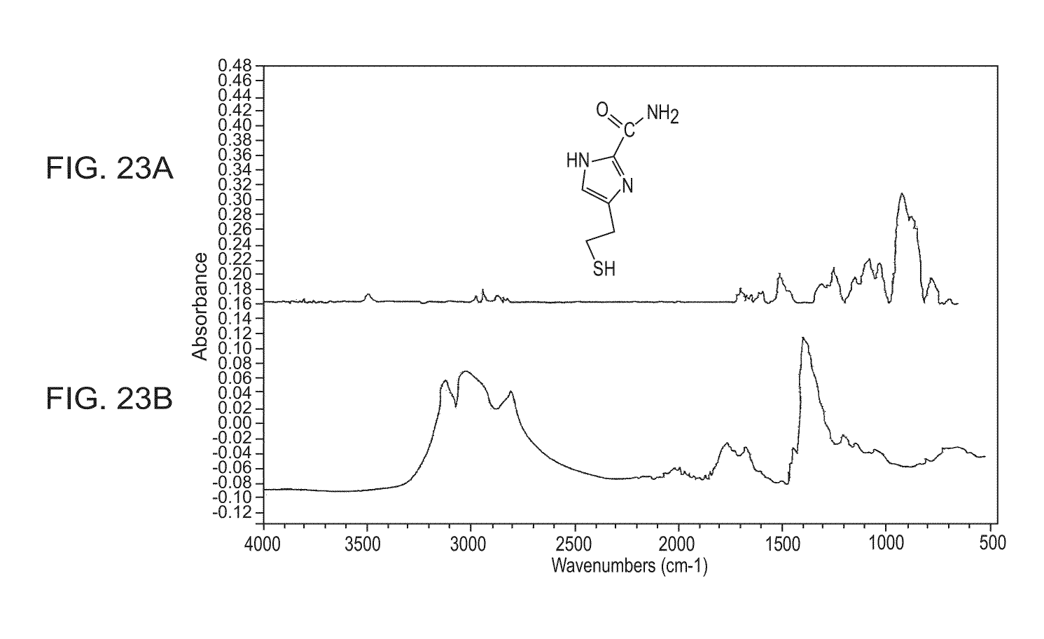

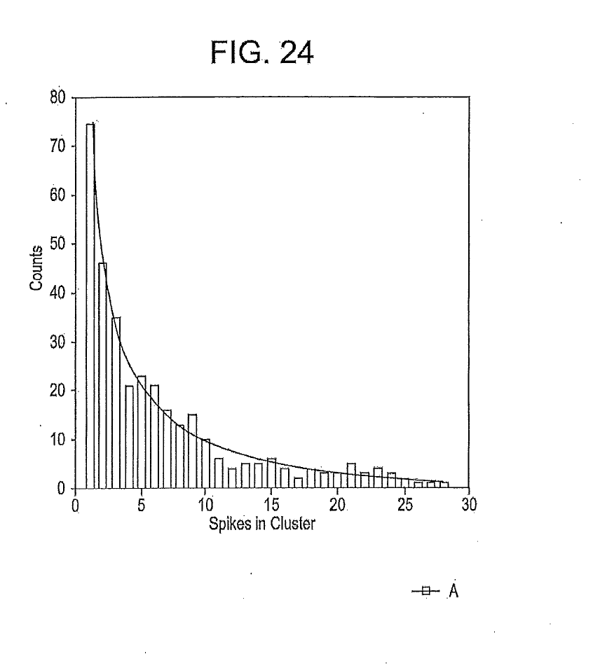

Recognition tunneling signals are not restricted to DNA bases. Accordingly, other molecules can be determined using recognition tunneling. For example, FIGS. 26A-26H show representative recordings from seven amino acids and FIGS. 27A-27C show recordings from small peptides (triglycine, GGG, tetraglycine, GGGG and a peptide containing both leucine and glycine, GGLL), generated according to various embodiments. Table 6 (below) shows the true positive rate with which individual signal spikes are called by the SVM according to an example.

TABLE 6
Example of true positive rates

| Amino Acid | TP Rate/Spike % | Majority/Cluster % | N |
|---|---|---|---|
| Whole Group | 75.5 (±3.5) | 77.7 (±3.5) | 171,654 |
| L-Arg | 72.1 (±3.5) | 78.8 (±3.5) | 17,129 |
| L-Asn | 78.2 (±3.5) | 80.2 (±3.5) | 26,921 |
| D-Asn | 70.5 (±3.5) | 72.9 (±3.5) | 28,176 |
| L-Leu | 71.4 (±3.5) | 71.7 (±3.5) | 21,648 |
| L-Ile | 71.7 (±3.5) | 77.6 (±3.5) | 20,356 |
| Gly | 75.4 (±3.5) | 77.3 (±3.5) | 30,470 |
| Me-Gly | 82.5 (±3.5) | 82.5 (±3.5) | 26,954 |
| Peptide | TP Rate/Spike % | Majority/Cluster % | |
| Gly-Gly-Gly | 95.7 (±5) | 95.7 (±5) | 947 |
| Gly-Gly-Gly-Gly | 79.0 (±3.5) | 79.0 (±3.5) | 12,247 |
| Gly-Gly-Leu-Leu | 91.3 (±3.5) | 91.3 (±3.5) | 12,352 |
| (Gly) | (73.0) | (74) | (30,470) |

In this example, true positive rate for each signal spike (TP Rate) and a majority vote within clusters (Majority) for all seven amino acids may be analyzed simultaneously. After training, 2,000 spikes were selected randomly from the total pool (N, right column) for testing. Errors were determined by repeating these tests on other randomly chosen blocks of 2,000 spikes. For this particular parameter combination (Table 6), about 10% of the spikes were not discriminated. Results for a pool of three peptides are listed below (GGG testing was limited to the 947 spikes recorded). Glycine (in parenthesis) was included in the pool to show how the amino acid signals are discriminated from the peptide signals.

In the example, the true positive rate called using cluster data (second column of Table 6) was based on a majority vote of the calls within each cluster. Because each cluster likely corresponds to a particular trapping geometry of a molecule in the tunnel junction, accuracy may not be much improved by this voting procedure (second column of Table 6). Accordingly, in the absence of these cluster correlations, the “majority vote” may be a powerful way to improve accuracy, because the probability of repeating a wrong call, p_{w}, is small and falls as on N successive wrong calls. Once spikes had been called by the SVM, cluster correlations are removed by randomizing their order and then applied a majority-voting algorithm to a sliding window containing an increasing number, N, of spikes.

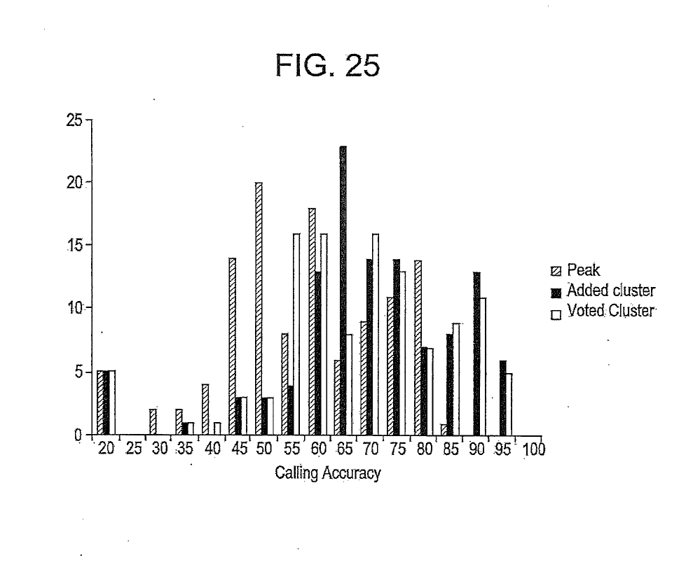

A true positive rate obtained for each of the seven amino acids as a function of N is shown in FIG. 28. In some embodiments, accuracies approach 100% with 3 to 20 spikes sampled, depending on the amino acid (for example). Such an algorithm may be limited to measurements in which the same analyte is sampled (for example, following chromatographic separation) but mixed samples may be analyzed using a hidden Markov model (for example) to take account of the correlations.

The robustness of the method was tested by repeating each of the measurements at least four times using new sample preparations and different tunnel junctions, with the SVM trained on a small (<3%) subset of the data.

The results show that recognition tunneling signals contain a large amount of information, as is clear from the complex, and very different pulse shapes shown in the insets in FIGS. 26A-26H. Table 6 demonstrates that a plurality of amino acids (e.g., 7) can be discriminated with accuracy, particularly when calls are improved using the majority voting algorithm based on blocks of randomized peaks (e.g., FIG. 28).

As to the number of analytes such embodiments may be applied to can be determined in the following manner. A correlation analysis was carried out among 40 parameters that characterize each signal spike, as listed in Table 7 (below).

TABLE 7
Starting parameters used in the signal analysis of amino acids

| Parameter | Description |
|---|---|
| ‘Amplitude’ | Max value of peak |
| ‘Average Amplitude’ | Average across whole peak |
| ‘Top Average’ | average of points 5 pts from start and end |
| ‘Peak Width’ | Full width at half height |
| ‘Roughness’ | standard deviation of peak |
| ‘Total Power’ | Integrated value of FFT power spectrum |
| | minus peak average (fluctuations) |
| ‘iFFT_L’ | Average of 3 points in middle of 1st FFT bin |
| ‘iFFT_M’ | Average of 3 points in middle of 4th FFT bin |
| ‘iFFT_H’ | Average of 3 points in middle of 9th FFT bin |
| ‘highLowRatio’ | ratio of FFT_9 to peak FT1 |
| ‘Peak_FFT_1’ | Average of FFT components in 1st frequency |
| | interal-avg peak pwr |
| ‘Peak_FT_2’ | Average of FFT components in 2nd frequency |
| | interval |
| ‘Peak_FT_3’ | Average of FFT components in 3rd frequency |
| | interval |
| ‘Peak_FT_4’ | Average of FFT components in 4th frequency |
| | interval |
| ‘Peak_FT_5’ | Average of FFT components in 5th frequency |
| | interval |
| ‘Peak_FT_6’ | Average of FFT components in 6th frequency |
| | interval |
| ‘Peak_FT_7’ | Average of FFT components in 7th frequency |
| | interval |
| ‘Peak_FT_8’ | Average of FFT components in 8th frequency |
| | interval |
| ‘Peak_FT_9’ | Average of FFT components in 9th frequency |
| | interval |
| ‘ClusterInfo.PeaksIn | Number of peaks in cluster |
| Cluster’ | |
| ‘Frequency’ | peaks per unit of time (no cluster boundaries) |
| ‘ClusterInfo.Average | Average amplitude across the cluster |
| Ampitude’ | |
| ‘ClusterInfo.Top | Largest amplitude averged across tops of |
| Amplitude’ | cluster |
| ‘ClusterInfo.Cluster | Cluster duration in samples (time) |
| Width’ | |
| ‘ClusterInfo.Roughness’ | RMS ampitude |
| ‘ClusterInfo.Ampitude’ | Maximum amplitude in cluster |
| ‘ClusterInfo.Total Power’ | Integrated value of FFT power spectrum |
| | minus peak average (fluctuations) |
| ‘ClusterInfo.iFFT_L’ | Average of 3 points in middle of 1st FFT bin |
| ‘ClusterInfo.iFFT_M’ | Average of 3 points in middle of 3rd FFT bin |
| ‘ClusterInfo.iFFT_H’ | Average of 3 points in middle of 6th FFT bin |
| ‘ClusterInfo.Peak_FFT_1’ | Average of FFT components in 1st frequency |
| | inter-avg peak pwr |

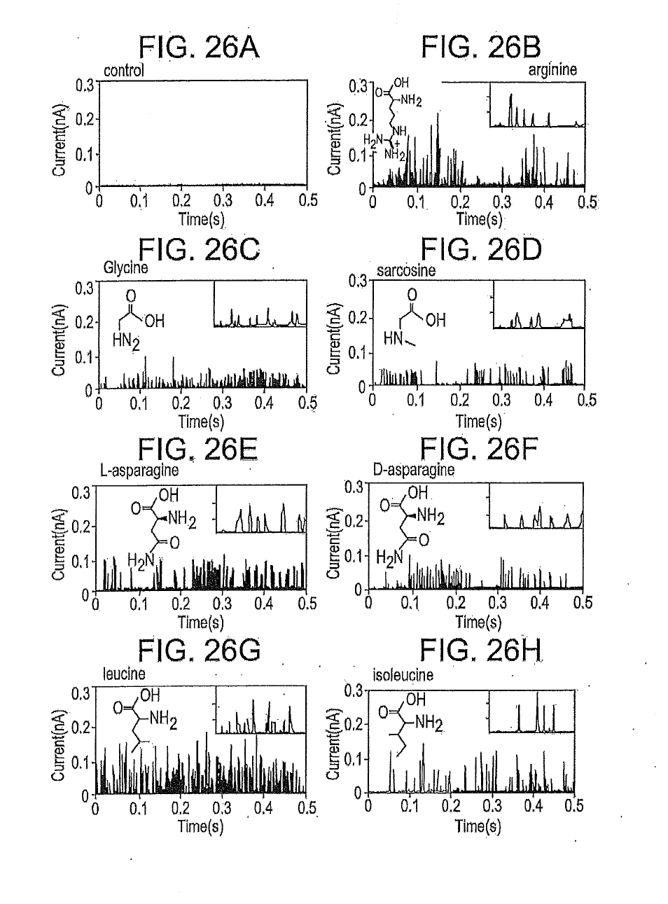

The correlation between different pairs of parameter sets (x,y) may be defined in the usual way, σxy= [symbol] (x− x̅)(y− y̅) [symbol] where the components were normalized using σₓₓ=1. Data from the pool was used to generate a correlation matrix where correlations are shown by off-diagonal elements. The matrix for the data for the seven amino acids can be found in FIG. 29, and the corresponding parameters are listed in Table 8 below. Trial and error resulted in rejecting all parameter combinations for which o_{xy}>0.7. One parameter from each correlated set was chosen for the final analysis.

TABLE 8
Parameters used to generate the correlation matrix (FIG. 29)

| Parameter | |
|---|---|
| Number | Parameter |
| 1 | ‘ClusterIndex’ |
| 2 | ‘PeakIndex’ |
| 3 | ‘Amplitude’ |
| 4 | ‘Average Amplitude’ |
| 5 | ‘Top Average’ |
| 6 | ‘Peak Width’ |
| 7 | ‘Roughness’ |
| 8 | ‘Total Power’ |
| 9 | ‘iFFT_L’ |
| 10 | ‘iFFT_M’ |
| 11 | ‘iFFT_H’ |
| 12 | ‘HighLowRatio’ |
| 13 | ‘Peak_FFT_1’ |
| 14 | ‘Peak_FFT_2’ |
| 15 | ‘Peak_FFT_3’ |
| 16 | ‘Peak_FFT_4’ |
| 17 | ‘Peak_FFT_5’ |
| 18 | ‘Peak_FFT_6’ |
| 19 | ‘Peak_FFT_7’ |
| 20 | ‘Peak_FFT_8’ |
| 21 | ‘Peak_FFT_9’ |
| 22 | ‘ClusterInfo.Peaks In |
| | cluster’ |
| 23 | ‘Frequency’ |
| 24 | ‘ClusterInfo.Average |
| | Amplitude’ |
| 25 | ‘ClusterInfo.Top |
| | Amplitude’ |
| 26 | ‘ClusterInfo.Cluster |
| | Width’ |
| 27 | ‘ClusterInfo.Roughness’ |
| 28 | ‘ClusterInfo.Amplitude’ |
| 29 | ‘ClusterInfo.Total Power’ |
| 30 | ‘ClusterInfo.iFFT_L’ |
| 31 | ‘ClusterInfo.iFFT_M’ |
| 32 | ‘ClusterInfo.iFFT_H’ |
| 33 | ‘ClusterInfo.Peak_FFT_1’ |
| 34 | ‘ClusterInfo.Peak_FFT_2’ |
| 35 | ‘ClusterInfo.Peak_FFT_3’ |
| 36 | ‘ClusterInfo.Peak_FFT_4’ |
| 37 | ‘ClusterInfo.Peak_FFT_5’ |
| 38 | ‘ClusterInfo.Peak_FFT_6’ |
| 39 | ‘ClusterInfo.FreqPeaks1’ |
| 40 | ‘ClusterInfo.FreqPeaks2’ |

This selection process resulted in the remaining seventeen nearly-independent parameters listed in Table 9 below.

TABLE 9
Independent parameters.

Surviving Parameters
‘Peak Width’
‘Total Power’
‘iFFT-L’
‘HighLowRatio’
‘Peak_FFT_1’
‘Peak_FFT_8’
‘Peak_FFT_9’
‘Freqeuncy’
‘ClusterInfo.Top Amplitude’
‘ClusterInfo.Roughness’
‘ClusterInfo.Amplitude’
‘ClusterInfo.Total Power’
‘ClusterInfo.iFFT_L’
‘ClusterInfo.iFFT_M’
‘ClusterInfo.Peak_FFT_4’
‘ClusterInfo.Peak_FFT_5’
‘ClusterInfo.FreqPeaks3’

In some embodiments, given the choice of upper limit of the correlation coefficient of 0.7, it may be possible to use binary discrimination, that is, assigning a parameter as high if it lies above 0.5 on a normalized scale (see below) and low if it lies between 0.5 to determine on the order of at least 2¹⁷ combinations (1.3×10⁵) of analytes. Thus, one of skill in the art will appreciate that a vast number of analytes may be discriminated according to embodiments of the present disclosure, yielding a powerful general analytical technique for analyzing molecules (e.g., single molecules).

In order not to bias the analysis towards parameters with bigger numerical values, parameters may be rescaled as follows: for each parameter value distribution measured for one amino acid (arginine for the amino acid analysis, glycine for the peptide analysis) the scale factor and additive constant were determined that moved the mean of the distribution to zero and the standard deviation to 1.0. The parameter values for all of the parameters for all of the other analytes may also be remapped using the same linear transformation. Thus, the means and standard deviations for each distribution may be scaled relative a renormalized set of values for one of the analytes in which each parameter has equal weight.

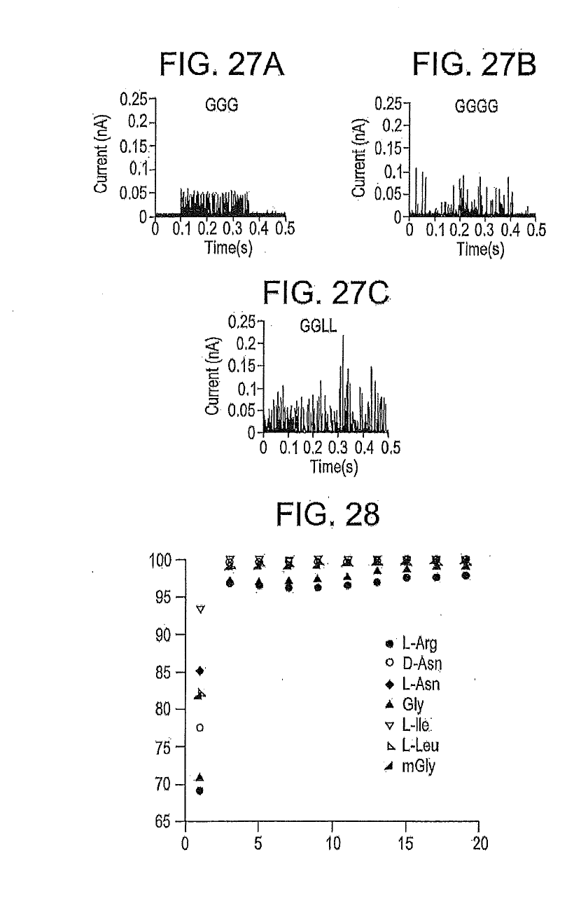

In practice, in some embodiments, particular parameters play roles in separating data. The specific parameters which may be dominate depend on a particular analyte. FIGS. 30A-30C show how just two or three parameters can provide significant discrimination between paired analytes, according to some embodiments. The variables used describe spike shape (Table 7). Significant discrimination between enantiomers (FIG. 30A) and isobaric isomers (FIG. 30B) may be obtained with just two parameters, while three parameters may be required to resolve glycine and sarcosine (FIG. 30C).

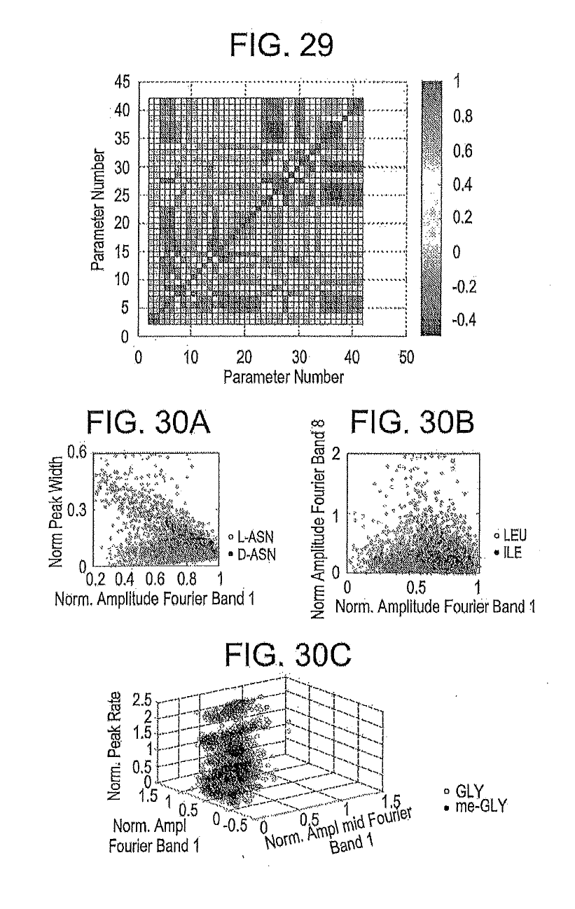

In another example, signals from mixed samples may also be complicated by interactions between the analytes. Accordingly, analysis of signal trains generated from mixtures of L- and D-asparagine using the same support vectors developed for the pure amino acid solutions may result in about half of the spikes not being recognized. This may imply that interactions between the enantiomers may have introduced new signals not seen in pure solutions. Nonetheless, spikes identified track the known composition, as shown by the plot of measured composition vs. actual composition for the enantiomers in FIG. 31. The fit includes a quadratic term consistent with association between the enantiomers. The solid line through the data points is given by

Rₘₑₐₛ=1.6R_{actual}−0.67R_{actual}²)

where

$$R = \frac{[ L ]}{[ L + D ]}$$

where [L] is the concentration of the L enantiomer and [L+D] is the total concentration of both. The actual ratio (R_{actual}) may be calculated from the measured input concentrations in the mixture and Rₘₑₐₛ is the ratio determined by taking the number of L calls made by the SVM and dividing it by the sum of the L- and D-calls.

The data is reproducible as shown by the repeated measurements. Such repeated measurements were made with freshly prepared samples with different tunnel junctions. However, it has been found that the SVM produces nearly identical results.

### Experimental Methods—According to Some Embodiments

Nucleoside 5′-monophosphates (from Sigma-Aldrich) were used as supplied. HPLC purified DNA oligomers were purchased from IDT. Tunneling measurements were carried out using gold probes and gold substrates. Gold probes were etched as described previously (Chang et al., 2010) and coated with high-density polyethylene (Tuchband et al, 2012; Visoly-Fisher et al, 2006) to leave a fraction of a micron of exposed gold. These probes gave no measureable DC leakage, important as this can be a source of distortion of the tunneling signal (Chang et al, 2010). Capacitative coupling of 120 Hz switching signals was an issue minimized by careful control of the coating profile. It was also diminished by functionalization of the probes.

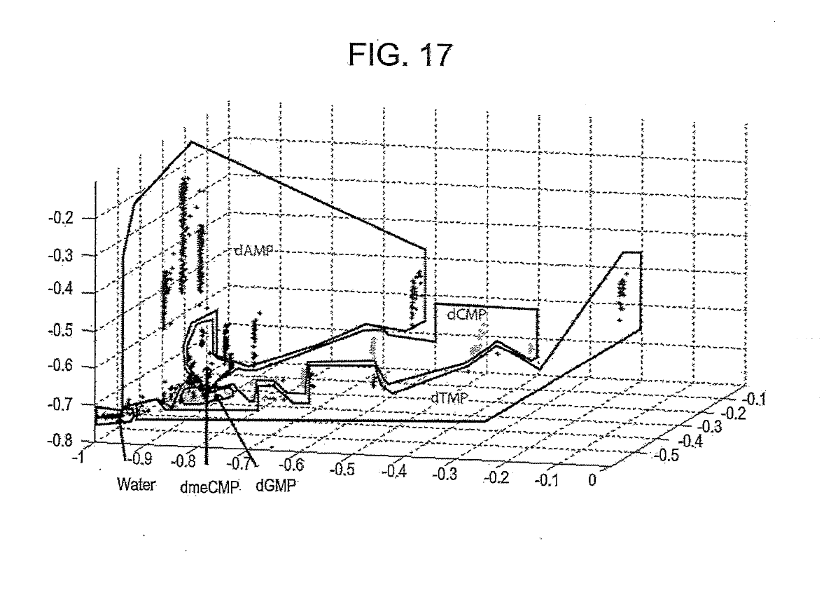

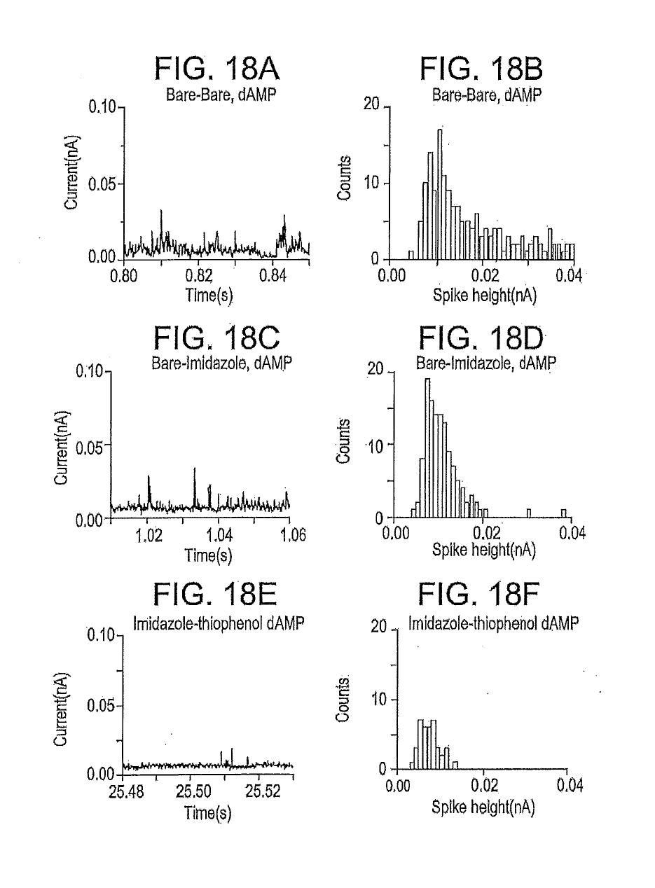

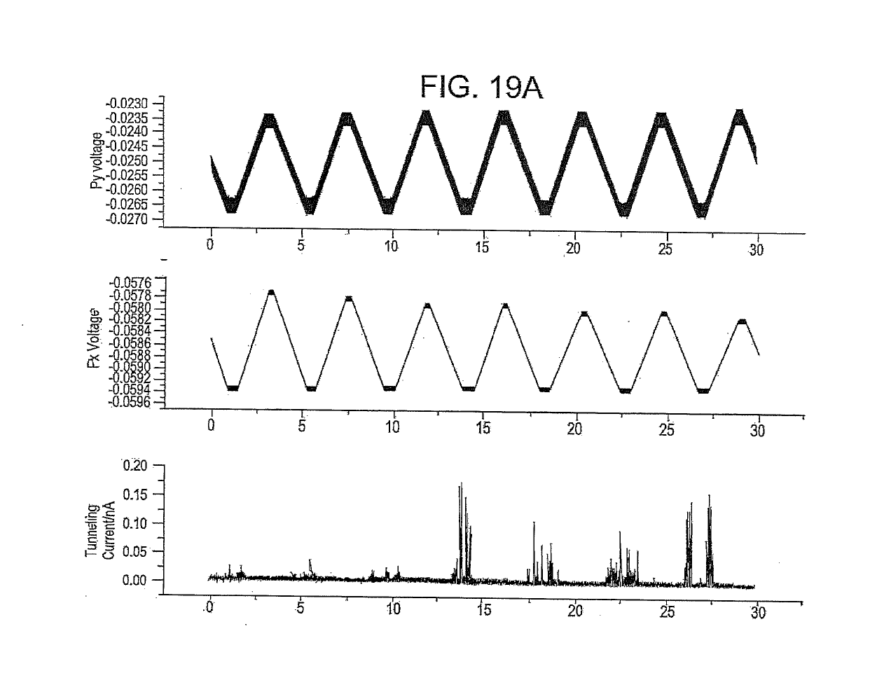

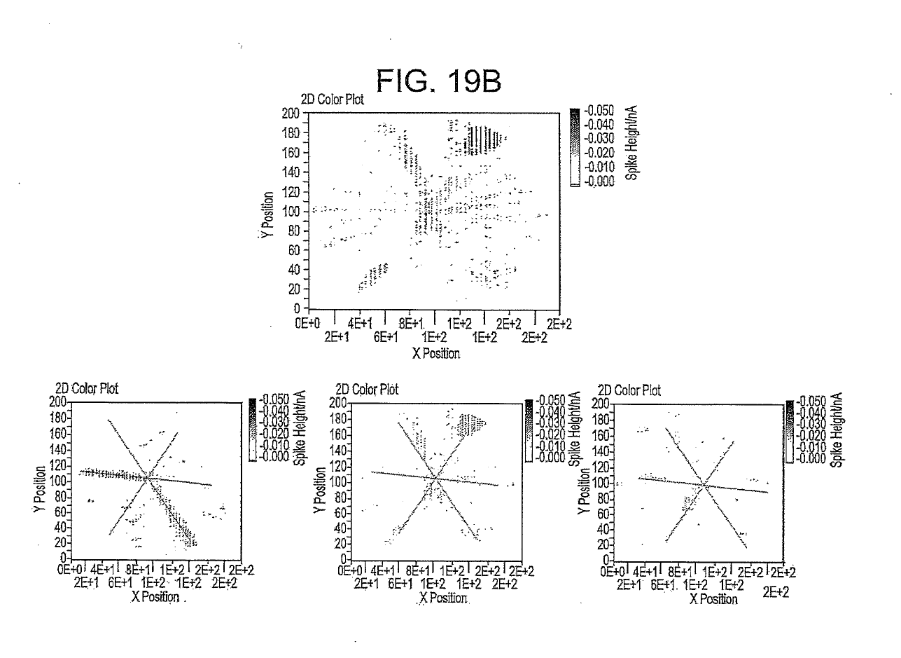

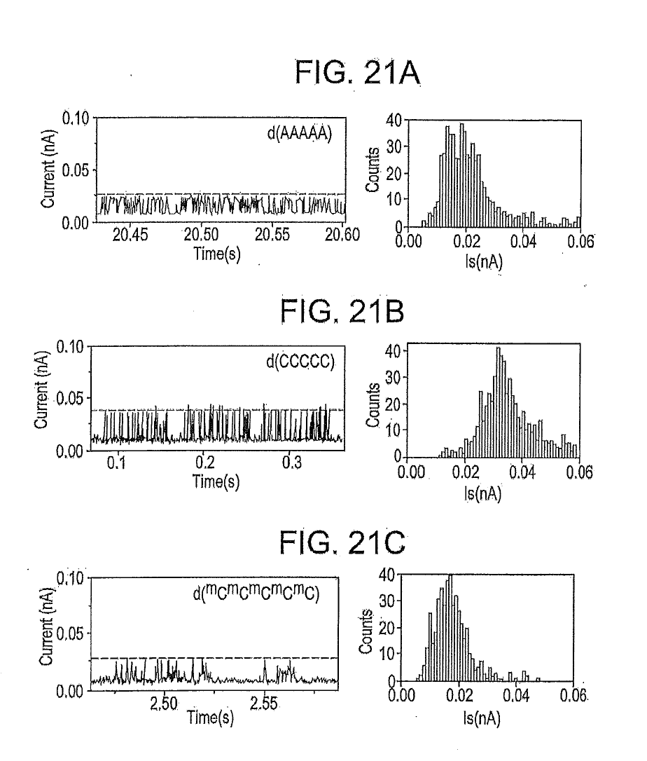

Gold (111) substrates (DeRose et al, 1993) were annealed with a hydrogen flame and then immediately immersed in a 2 mM ethanol solution of 4(5)-(2-thioethyl)-1H-imidazole-2-carboxamide (Liang et al. 2011), where they were left for a minimum of 2 h (usually overnight), then rinsed in ethanol and blown dry with nitrogen before immersion in the phosphate buffer solution. Characterization of the resulting monolayers is described in FIGS. 23A and 23B. Insulated probes were cleaned prior to functionalization by rinsing with ethanol and H₂O, blown dry with nitrogen gas, and then immersed in a 1 mM methanolic solution of 4(5)-(2-thioethyl)-1H-imidazole-2-carboxamide (Liang et al. 2011) in methanol for 1 h. The efficiency of the functionalization process may be tested by making recognition tunneling measurements on a functionalized gold surface, and comparing the tunneling data to controls in which the probe was functionalized, however, in an analysis, the substrate was left bare. The resulting tunneling signals indicate whether or not functionalization was successful (FIGS. 18A-18F).

Current signals were recorded using an Agilent PicoSPM (Agilent Chandeler, Ariz.) together with a digital oscilloscope controlled by a custom Labview program. The servo response time was set to about 30 ms as described previously (Chang et al, 2010). This places an upper limit on undistorted measurements of pulse widths of a few ms.

The “clock-scanning” system was developed around a Field-Programmable Gate Array (FPGA). A computer running Lab View (Version 8.5.1, National Instruments) controlled the FPGA as well as issued API calls to Pico View (Version 1.8, Agilent, Chandler, Ariz.) via PicoScript (Beta Version, Agilent, Chandler, Ariz.). For experiments where the tip was moving at a specified speed the tip was set to an initial location from the LabView interface. A radius around this position was set along with a desired tip speed. The tip was then moved in a spoke pattern around the initial point changing by a user specified number of degrees, by issuing tip movement commands to PicoView. The FPGA (PCIe-7842R, National Instruments) contains a built in A/D that enabled the tunneling signal to be recorded at 50 kHz from the breakout box. The position of the tip was also recorded by using a voltage divider and reading the piezo voltages for the x and y directions from the breakout box. Provision was made in the code for enabling and disabling the servo at selected point on the scan, and for leveling the orientation of the scan with respect to the substrate as described above.

As described above, a support vector machine (SVM) may be used to identify one or more molecules from data generated in a recognition tunneling (RT) apparatus. The SVM can achieve a relatively high accuracy by using a plurality of parameters to be able to identify a molecule that generated a particular signal. In some embodiments of the present disclosure, the accuracy of calling the correct molecule from data produced by an RT apparatus may be increased using, for example, merely two parameters, if such parameters are used together.

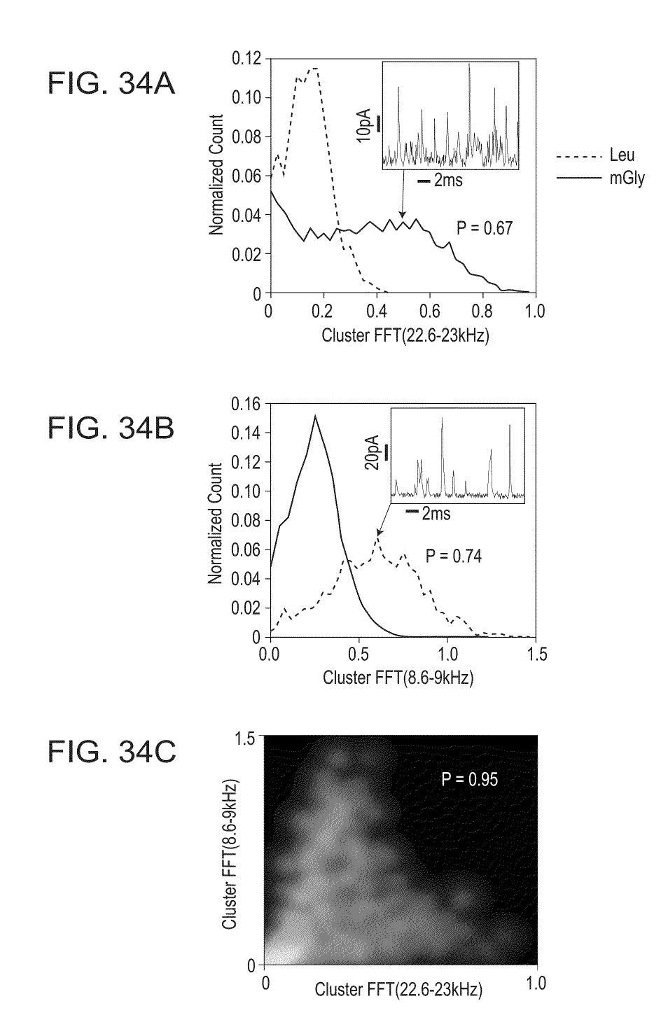

For example, FIG. 34A shows distributions of the values of a Fast Fourier transform (FFT) amplitudes for signal clusters in the frequency range of 22.6-23 kHz for two analytes in the illustrated case—methylglycine and leucine. FIG. 34B shows the distribution of the values of FFT amplitudes for signal clusters in the frequency range of 8.6 to 9 kHz for the same two analytes. The insets of each figure show signal trains representative of values pointed to by the arrows—signals with higher FFT amplitudes at high frequency are sharper (see, e.g., inset FIG. 34A).

Since the distributions of the two types of FFT amplitudes are different, each parameter can then be used to determine from which analyte the amplitudes corresponds to. For example, if all events with amplitudes above 0.3 of the FFT in the 22.6-23 kHz (FIG. 34A) range are called to correspond to methylglycine, and all events with amplitudes below 0.3 of the FFT are called to correspond to as leucine, this leads to a correct call 67% of the time. A similar analysis applied to the data in FIG. 34B would call the analyte correctly 74% of the time.

In some embodiments of the present disclosure, the accuracy of calling the correct molecule may be increased upon using the two parameters together. For example, in some embodiments, a method of assigning a chemical identity to a molecule signal is provided, where the method may comprise one or more (in some embodiments, several, and in some embodiments, all) of the following steps: collecting signal data for at least two different molecules from a molecular identification or sequencing apparatus, the data including information corresponding to at least two signal parameters, determining the distribution of the frequency of occurrence of the values of each of the parameters, creating a plurality of at least three-dimensional plots, wherein each plot comprises the determined values for a pair of parameters, such that, the determined values for each parameter is plotted versus each of the other remaining parameters, determining the separation of values between different analyte molecules for each of the plots, selecting at least one plot of the plurality of plots which includes a separation of values between the two analyte molecules greater than a predetermined amount, and determining the identity of signals according to their determined value location on the selected plot.

In such embodiments, selecting at least one plot comprises selecting only a single plot, and the single plot is selected based on the separation of values between the two molecules being the greatest among the plurality of plots.

In some embodiments, a method of assigning a chemical identity to a molecule signal, is provided, where the method may comprise one or more (in some embodiments, several, and in some embodiments, all) of the following steps: measuring a plurality of distributions of two or more signal parameters from signal data collected from a molecular identification or sequencing apparatus for known molecules, determining at least one pair of parameters that best determine the separation of signals for identifying the known molecules, and using the pair of determined parameters, identifying one or more unknown molecules from second signal data collected from a molecular identification or sequencing apparatus.

Other embodiments include a system for assigning a chemical identity to a molecule signal, where the system comprises data collection means (e.g., a computer and/or the like) configured to collect signal data for at least two different molecules from a molecular identification or sequencing apparatus, the data including information corresponding to at least two signal parameters, and at least one processor having computer code operational thereon configured for: determining the distribution of the frequency of occurrence of the values of each of the parameters, creating a plurality of at least three-dimensional plots, wherein each plot comprises the determined values for a pair of parameters, such that, the determined values for each parameter is plotted versus each of the other remaining parameters, determining the separation of values between different analyte molecules for each of the plots, selecting at least one plot of the plurality of plots which includes a separation of values between the two analyte molecules greater than a predetermined amount, and determining the identity of signals according to their determined value location on the selected plot. As noted in the related method embodiments, selecting at least one plot may comprise selecting only a single plot, and the single plot may be selected based on the separation of values between the two molecules being the greatest among the plurality of plots.

Still other embodiments include a system of assigning a chemical identity to a molecule signal which comprises at least one computer processor having computer code operational thereon configured for: measuring a plurality of distributions of two or more signal parameters from signal data collected from a molecular identification or sequencing apparatus for known molecules, determining at least one pair of parameters that best determine the separation of signals for identifying the known molecules, and using the pair of determined parameters, identifying one or more unknown molecules from second signal data collected from a molecular identification or sequencing apparatus.

Accordingly, examples are detailed below, with reference to the figures.

As shown in FIG. 34C, a three dimensional histogram plots the values from FIG. 1a along the horizontal axis, and the values from FIG. 1b along the vertical axis with the frequency represented by the density of red (methylglycine) or green (leucine) spots. The overlap area (yellow region) is reduced to a small area near the origin (0,0), and illustrates that the two analytes can be separated (i.e., correctly called) to 95% accuracy on each single molecule signal.

To that end, in some embodiments of the present disclosure, any signal train from a single molecule sensing apparatus (e.g., an RT apparatus) may be used this way. For example, an ion current passed though a nanopore could be used where the parameters are the size (for example) of the ion current blockade and the width of the blockade signal. The parameters, however, could include (for example) the RMS noise on the signal, FFT components of the transform of the peaks, distributions of levels within peaks and the like.

In some embodiments, the analysis may proceed as follows. A multi-parameter SVM analysis is carried out. Thereafter, the analysis is repeated with the weight of a given parameter reduced in turn. Parameters that cause the largest loss of accuracy are assigned as the most significant parameters (as was the case as to how the two FFT components were identified in the data shown in FIGS. 34A-34C). Selected parameters (in some embodiments, the top or best few parameters (e.g., two)) can then be plotted against the other to determine which combination produces the best pairwise separation of the data.

Various implementations of the embodiments disclosed above, in particular at least some of the methods/processes disclosed, may be realized in digital electronic circuitry, integrated circuitry, specially designed ASICs (application specific integrated circuits), computer hardware, firmware, software, and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

Such computer programs (also known as programs, software, software applications or code) include machine instructions for a programmable processor, for example, and may be implemented in a high-level procedural and/or object-oriented programming language, and/or in assembly/machine language. As used herein, the term “machine-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the subject matter described herein may be implemented on a computer having a display device (e.g., a CRT (cathode ray tube) or LCD (liquid crystal display) monitor and the like) for displaying information to the user and a keyboard and/or a pointing device (e.g., a mouse or a trackball) by which the user may provide input to the computer. For example, this program can be stored, executed and operated by the dispensing unit, remote control, PC, laptop, smart-phone, media player or personal data assistant (“PDA”). Other kinds of devices may be used to provide for interaction with a user as well; for example, feedback provided to the user may be any form of sensory feedback (e.g., visual feedback, auditory feedback, or tactile feedback); and input from the user may be received in any form, including acoustic, speech, or tactile input.

Certain embodiments of the subject matter described herein may be implemented in a computing system and/or devices that includes a back-end component (e.g., as a data server), or that includes a middleware component (e.g., an application server), or that includes a front-end component (e.g., a client computer having a graphical user interface or a Web browser through which a user may interact with an implementation of the subject matter described herein), or any combination of such back-end, middleware, or front-end components. The components of the system may be interconnected by any form or medium of digital data communication (e.g., a communication network). Examples of communication networks include a local area network (“LAN”), a wide area network (“WAN”), and the Internet.

The computing system according to some such embodiments described above may include clients and servers. A client and server are generally remote from each other and typically interact through a communication network. The relationship of client and server arises by virtue of computer programs running on the respective computers and having a client-server relationship to each other.

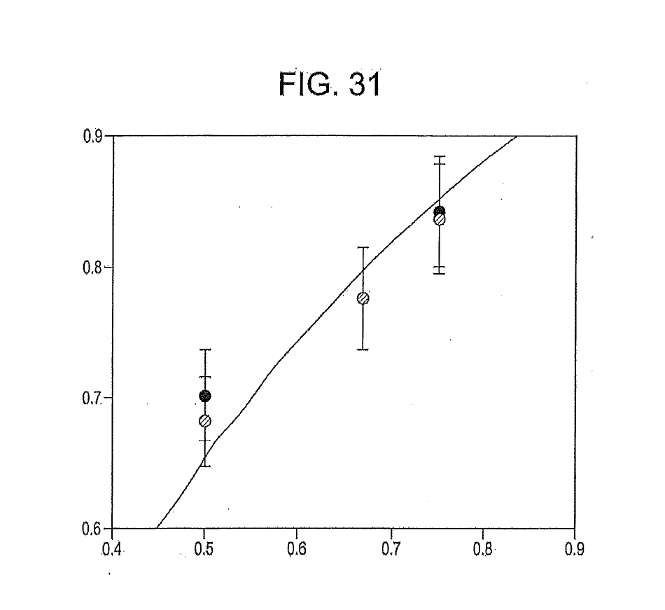

For example, as shown in FIG. 32 at least one processor which may include instructions operating thereon for carrying out one and/or another disclosed method, which may communicate with one or more databases and/or memory—of which, may store data required for different embodiments of the disclosure. As noted, the processor may include computer instructions operating thereon for accomplishing any and all of the methods and processes disclosed in the present disclosure. Input/output means may also be included, and can be any such input/output means known in the art (e.g., display, printer, keyboard, microphone, speaker, transceiver, and the like). Moreover, in some embodiments, the processor and at least the database can be contained in a personal computer or client computer which may operate and/or collect data. The processor also may communicate with other computers via a network (e.g., intranet, internet).

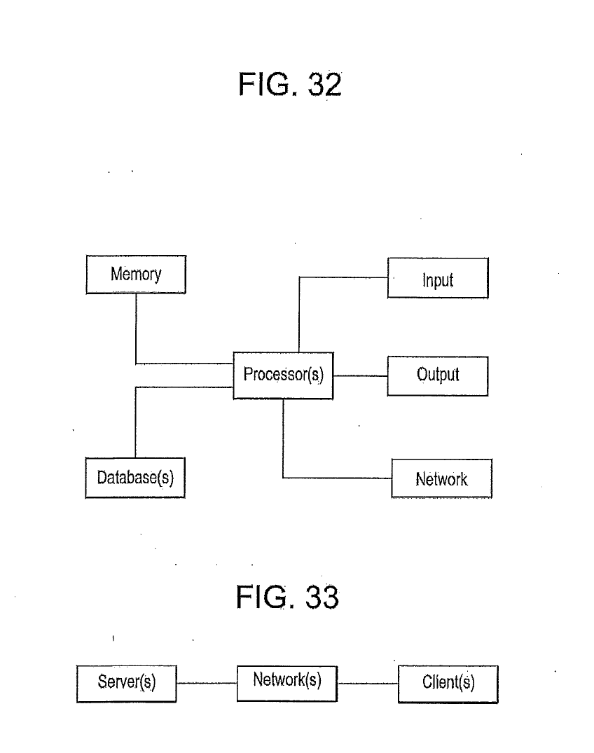

Similarly, FIG. 33 illustrates a system according to some embodiments which may be established as a server-client based system, in which the client computers are in communication with databases, and the like. The client computers may communicate with the server via a network (e.g., intranet, internet, VPN).

Any and all references to publications or other documents, including but not limited to, patents, patent applications, articles, webpages, books, etc., presented in the present application, are herein incorporated by reference in their entirety.

Although a few variations have been described in detail above, other modifications are possible. For example, any logic flow depicted in the accompanying figures and described herein does not require the particular order shown, or sequential order, to achieve desirable results. Other implementations may be within the scope of at least some of the following exemplary claims.

Example embodiments of the devices, systems and methods have been described herein. As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements from any other disclosed methods, systems, and devices, including any and all elements corresponding to methods, systems and devices for improving the accuracy of chemical identification in a recognition tunneling junction. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure).

### REFERENCES

The following references, to the extent that they provide exemplary procedural or other details supplementary to those set forth herein, are specifically incorporated herein by reference.
Branton et al, Nature Biotech., 26: 1146-1153, 2008.
Chang et al, J. Am. Chem. Soc, 133: 14267-14269, 2011.
Chang et al, Nano Lett., 10: 1070-1075, 2010.
Chang et al, Nanotech., 20: 195102-185110, 2009.
Clarke et al, Nature Nanotech., 4:265-270, 2009.
DeRose et al, Vac. Sci. Techno!., Al 1:776-780, 1993.
Derrington et al, Proc. Natl Aca. Sci, USA, 107: 16060-16065, 2010.
Friddle et al, Phys. Chem. C, 1 12:4986-4990, 2008
Fuhrmann et al, Biophysical J, 2011 (submitted)
Huang et al, Nature Nanotech., 5:868-873, 2010.
Liang et al, Chemistry, 2011 (submitted)
Lindsay et al, Nanotech., 21:262001-262013, 2010.
Pathak et al, Applied Physics Lett., 100:023701, 2012.
Saha et al, Nano Lett., 12:50-55, 2012.
Tsutsui et al, Nature Nanotech., 5:286-290, 2010.
Tsutsui et al, Nature Sci. Rept., 1:46, 2011.
Tuchband et al, Rev. Sci. Instrum., 83:015102, 2012.
Visoly-Fisher et al, Proc. Natl. Aca. Sci, USA, 103:8686-8690, 2006.
Zwolak and Di Ventra, Nano Lett., 5:421-424, 2005.
Zwolak and Di Ventra, Rev. Modern Physics, 80: 141-165, 2008.

1. A method of assigning the identity of signals generated by electron tunneling through an analyte, the method comprising:
determining a plurality of characteristics of each signal spike;
generating one or more training signals with a set of analytes comprising at least a first analyte and a second analyte; and
using the training signals to find one or more boundaries in a space of dimension equal to one or more parameters, wherein the space is partitioned such that a signal from the first analyte of interest is separated from a signal from the second analyte of interest.

2. The method of claim 1, wherein the number of boundaries are less than or equal to the number of parameters.

3. The method of claim 1, wherein the set of analytes contains more than two analytes.

4. The method of claim 1, wherein the one or more parameters describes relationships between successive spikes.

5. The method of claim 1, wherein the one or more parameters are obtained from a Fourier analysis of the spikes.

6. The method of claim 1, wherein the one or more parameters are obtained from a Wavelet analysis of the spikes.

7. The method of claim 1, wherein the one or more parameters are obtained from a Fourier analysis of clusters of spikes.

8. The method of claim 1, wherein the analytes include at least one of DNA bases, modified DNA bases, amino acids, or modified amino acids.

9-11. (canceled)

12. The method of claim 1, further comprising weighting the calls by the frequency with which a call is repeated within a cluster of signals.

13. The method of claim 1, wherein training is accomplished using a support vector machine.

14. The method of claim 1, in which the parameter set is reduced by removing one of each pair of parameters for which the correlation coefficient is 0.5 or higher.

15. The method of claim 1, in which the mean and range of parameter values are scaled by the same scale factors that normalize the parameter values of a chosen standard analyte.

16. A method for improving the accuracy of the identity of an analyte as called by the method of claim 1, whereby calls are made on a random sample of two or more calls, or on a random sample of two to about twenty calls.

17. A molecular spectroscopy in which electrical pulses generated by electron tunneling through analytes are characterized by a plurality of parameters, wherein the number of parameters is first reduced by rejecting one of each correlated pair, and then called using a machine learning algorithm previously trained with known samples.

18. A computer system for assigning the identity of signals generated by electron tunneling through an analyte, and/or improving the accuracy of the identity of an analyte, the system comprising at least one processor, wherein the processor includes computer instructions operating thereon for performing the steps of a method for assigning the identity of signals generated by electron tunneling through an analyte, and/or improving the accuracy of the identity of an analyte, according to any previous method claim.

19. A computer system for determining the identity of one or more analytes, and/or improving the accuracy of the identity of an analyte, comprising at least one processor, wherein the processor includes computer instructions operating thereon for performing the steps of a method for determining the identity of one or more analytes, and/or improving the accuracy of the identity of an analyte, utilizing a current versus time signal having three or more parameters.

20-26. (canceled)

27. A system of assigning a chemical identity to a molecule signal, the system comprising:
at least one computer processor having computer code operational thereon configured for:
measuring a plurality of distributions of two or more signal parameters from signal data collected from a molecular identification or sequencing apparatus for known molecules;
determining at least one pair of parameters that best determine the separation of signals for identifying the known molecules; and
using the pair of determined parameters, identifying one or more unknown molecules from second signal data collected from a molecular identification or sequencing apparatus.
